(19) United States
(12) Patent Application Publication
(10) Pub. No.: US 2005/0037484 A1
(43) Pub. Date: Feb. 17, 2005

(54) Optical bio-discs including spiral fluidic circuits for performing assays

(76) Inventors: Norbert Staimer, Lake Forest, CA (US); YihFar Chen, Kaohsing City (TW); James R. Norton, Santa Ana, CA (US); Jay O.C. Jison, Aliso Viejo, CA (US); Johnny Chen Mounphoxay, Anaheim, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/828,732

(22) Filed: Apr. 21, 2004

Related U.S. Application Data
(60) Provisional application No. 60/464,869, filed on Apr. 23, 2003.

(51) Int. Cl. C12M 1/34

(52) U.S. Cl. 435/287.2; 422/58

(57) ABSTRACT

The present invention relates to methods and apparatus for assays including optical bio-discs with spiral fluidic circuits and related detection systems. The optical bio-disc 110 includes a cap portion 116 having inlet and vent ports formed therein, a first channel layer 632 having cut-out portions, a second channel layer 634 having cut-out portions; a third channel layer 636 having cut-cut portions, a fourth channel layer 638 having cut-out portions, and a substantially circular substrate having a center and an outer edge. The cut-out portions are in register with each other such that when the bio-disc 110 is assembled a spiral fluidic circuit is formed having an inlet port, a mixing chamber 134, upper flow chambers 620, lower pass through chambers 622, inlet passages 626, outlet passages 628, a circumferential analysis chamber 618, and vent ports in fluid communication.

### BACKGROUND OF THE INVENTION

1. Field of the Invention

This invention relates in general to biochemical assays. More specifically, embodiments of the invention relate to methods and apparatus for assays including optical bio-discs with spiral fluidic circuits and related detection systems.

2. Description of the Related Art

The detection and quantification of analytes in the blood or other body fluids may be important for diagnosis of diseases, elucidation of the pathogenesis, and for monitoring the response to drug treatment. Traditionally, diagnostic assays are performed in laboratories by trained technicians using complex apparatus. Performing these assays is usually time-consuming and costly. Thus, there is a significant need to make diagnostic assays and forensic assays of all types faster and more local to the end-user. Ideally, clinicians, patients, investigators, the military, other health care personnel, and consumers should be able to test themselves for the presence of certain risk factors or disease indicators in their systems, and to test for the presence of certain biological material at a crime scene or on a battlefield. At present, there are a number of medical diagnostic, silicon-based, devices with nucleic acids and/or proteins attached thereto that are commercially available or under development. These chips are not for use by the end-user, or for use by persons or entities lacking very specialized expertise and expensive equipment.

The Optical Bio-Disc, also referred to as Bio-Compact Disc (BCD), bio-optical disc, optical analysis disc or compact bio-disc, is known in the art for performing various types of bio-chemical analyses. In particular, this optical disc utilizes the laser source of an optical storage device to detect biochemical reactions on or near the operating surface of the disc itself. These reactions may be occurring in small channels or chambers inside the disc, frequently with one or more dimensions of less than 500 microns, or may be reactions occurring on the open surface of the disc. Whatever the system, multiple reaction sites are usually needed either to simultaneously detect different reactions, or to repeat the same reaction for error detection purposes.

### SUMMARY OF THE INVENTION

The present invention relates to performing assays on an optical bio-disc including preparation and detection of genetic material, immuno-chemical assays, and colorimetric assays. The present invention also relates to chromatographic analysis on optical bio-discs, including for example, affinity, size exclusion, reverse phase, and ion exchange chromatography. Ion exchange chromatography may include anion exchange, cation exchange, cation exchange linked immunoassays (CELIA), and anion exchange linked immunoassays. These chromatographic assays may be performed in conjunction with calorimetric and/or fluorescent detection and quantitation using an optical analysis disc or optical bio-disc. The invention includes methods for preparing assays, methods for depositing the reagents for the assays, discs for performing assays, and detection systems.

High pressure liquid chromatography (HPLC) and other types of chromatography is generally used to separate substances or analytes of interest having different physical properties and quantitate these analytes using UV/VIS, IR, luminescence, or fluorescence detection. Chromatographic instruments generally require costly equipment and maintenance and trained personnel to carry out complicated time-consuming tests. It is an object of the present invention to make possible a simple chromatography system for testing analytes, portable and for use by the end user.

The present invention includes methods for isolating and quantifying the concentration of an analyte of interest in a biological sample on optical bio-discs using colorimetric or fluorometric detection. Analytes may include, for example, Hemoglobin, glycated and non-glycated hemoglobin, and other isoforms of proteins. All reagents necessary for the assays may be immobilized on the optical disc prior to the assay. To perform an assay, a sample (preferably serum, but other types of body fluids could also be used) is loaded into a channel or fluidic circuit through an injection or inlet port. After the sample in loaded, the inlet port is sealed, the disc is spun, and the sample is moved through one or more micro-chromatographic or biological matrices, by centrifugation, comprising different separation media including, for example, size exclusion and ion exchange matrices. The matrix may be formed from resins or beads, gels, or membranes. Once the analyte of interest is separated chromatographically, the analyte solution, containing the analyte of interest is then directed into an analysis chamber. The analysis chamber may contain detection reagents including, but not limited to, capture agents bound to the surface of a capture zone and signal antibodies conjugated with one or more reporters, both of which have affinity to different epitopes on the same analyte of interest. Reporters may include, but are not limited to, fluorophores, luminophores, microspheres, enzymes, and nanospheres. The analyte is incubated in the analysis chamber at a pre-determined temperature and time to allow sufficient binding of the analyte to the capture agent and binding of the signal antibodies to the analyte. After incubation the analysis chamber is washed to remove unbound signal antibodies and analytes. If the reporter used in the assay is a non-enzyme detectable reporter such as beads, then the analysis chamber may then be analysed for presence and amount of reporter beads using an optical disc reader. Otherwise, if an enzyme reporter is used, an enzyme substrate is added to the analysis chamber. The enzyme is allowed to catalize an enzyme-substrate reaction that produces a detectable signal such as color or fluorescence. The optical disc reader then quantifies the intensity of the color or fluorescence developed. In one embodiment, after approximately 3 minutes of data collection and processing, the results of the assay are displayed on a computer monitor. Alternatively, an inherent enzymatic activity of the analyte itself may be advantageously used to produce a detectable signal. A non-limiting example of such an analyte is hemoglobin that has an inherent peroxidase activity. Thus, capture and signal agents may not be necessary with this method, thereby allowing a one step assay method without the need for washing steps. In this method, the sample is loaded into the disc, ran through the matrix, and into the analysis chamber, as described above. The analysis chamber, in this method, would only contain the appropriate substrate, a peroxidase substrate like ABTS (2,2′-azino-di-[3-ethyl-benzthiazoline]sulfonic acid) may be used in conjunction with the hemoglobin analyte, for example. Once the signal is generated, the analysis chamber is investigated using the optical disc reader, as described above, to determine the presence and amount of analyte present in the sample.

It should be noted that some diagnostic colorimetric assays in clinical laboratories are carried out at 37 degrees Celsius to facilitate and accelerate color development. However, colorimetric assays may be carried out at any suitable temperature and, in some embodiments, calorimetric assays are performed on optical discs and are optimized to run at ambient temperature. The optimization includes selection of enzyme sources, enzymes concentrations, and sample preparation.

In one embodiment, various chromagens may be selected, for use in a calorimetric assay, where each chromagen may be detected by an optical reader at a specific wavelength. CD-R type disc readers, for example, may detect chromagens that are in the infrared region (750 nm to 800 nm). Other types of optical disc systems may be used in the present invention including DVD, DVD-R, fluorescent, phosphorescent, and any other similar optical disc reader. The amplitude of optical density measurements depends on the optical path length, the molar extinction coefficient of the chromagen and the concentration of the analyte of interest (Beer's law). To optimize the sensitivity of colorimetric assays on optical discs, several chromagens with high molar extinction coefficients at the wavelengths of interest have been identified and evaluated.

Chromagens suitable for calorimetric assays on CD-R type optical discs include, but are not limited to, N,N′-Bis(2-hydroxy-3-sulfopropyl)tolidine, disodium salt (SAT-3), N-(Carboxymethylaminocarbonyl)-4,4′-bis(dimethylamino)-diphenylamine sodium salt (DA-64), 2,2′-azino-dimethylthiozoline-6-sulfonate (ABTS), Trinder's reagents N-Ethyl-N-(2-hydroxy-3-sulfopropyl)3-methylaniline, sodium salt, dihydrate (TOOS) with the coupling reagent 3-(N-Methyl-N-phenylamino)-6-aminobenzenesulfonic acid, and sodium salt (NCP-11).

According to one aspect of the present invention, there are provided detection methods for quantifying the concentration of an analyte of interest in a biological sample on the bio-discs. The detection includes directing a beam of electromagnetic energy from a disc drive toward the capture field or zone, analysis chamber, or the bio-matrix materials and analyzing electromagnetic energy returned from or transmitted through.

The optical density change, in the colorimetric assay aspect of the present invention, may be quantified by the optical disc reader in at least two ways. These include measuring the change in light either reflected or transmitted. The disc may be referred to as reflective, transmissive, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light. Different detection systems are used for different types of bio-discs (top versus bottom detector).

The apparatus and methods in embodiments of the present invention can be designed for use by an end-user, inexpensively, without specialized expertise and expensive equipment. The system can be made portable, and thus usable in remote locations where traditional diagnostic equipment may not generally be available.

Alternatively, fluorescent assays can be carried out to quantify the concentration of an analyte of interest in a biological sample on the optical discs. In this case, the energy source in the disc drive preferably has a wavelength controllable light source and a detector that is or can be made specific to a particular wavelength. In yet another alternative, a disc drive can be made with a specific light source and detector to produce a dedicated device, in which case the source may only need fine-tuning.

Analysis of biological fluids aimed at the quantitative and qualitative determination of substances associated with a wide variety of physiological disorders, bio- research, proteomics, environmental studies, agriculture, and food industry, relies on specific binding assays from which the immunoassay plays a dominant role. The outstanding specificity and sensitivity for quantitative determination of an almost limitless number of analytes in practically any milieu, and the ability to miniaturize and adapt to automation makes them ideal tools for routine assays.

Antibody binding techniques are based on the interaction of a binding antibody, receptor, or other binding proteins with an antigen or a specific ligand molecule and the formation of an antibody-antigen or receptor-ligand complex. By changing certain conditions a binding assay can be designed to determine either an analyte, ligand, or target binding reagent or an antibody of interest. The steps are similar but the assay configuration provides results pertinent to the antigen or antibody of interest.

Capture Probe Binding and Sample Application

When a sample is injected into a micro-channel, fluidic circuit, or flow channel on an optical bio-disc, the target agent or Analyte, including, for example, target antigen or antibody, binds to a capture probe bound in a capture or target zone on a solid support such as a disc substrate or a bio-matix. The capture probe may be an antigen recognized by the target antibody or an antibody or receptor with specific affinity to the target antigen or ligand. Following the binding step, unbound target agent is removed through a wash step. It should be understood that various techniques, procedures and chemistries, know in the art, may be used to bind the capture probe onto a solid support, including, for example, direct covalent binding of probes onto a metallic or activated surface, passive adsorption, and through cross-linking reagents.

In addition to surface chemistries for attaching capture probes, blocking agents may be used to block areas within the capture or target zone and the flow channel where capture probes are not bound (non-capture areas) to prevent non-specific binding of the target or analyte, signal probes, and reporters onto these areas. Blocking agents include, but are not limited to proteins such as BSA, gelatin, sugars such as sucrose, detergents such as tween-20, genetic material such as sheared salmon sperm DNA, and polyvinyl alcohol.

Signal Generation

Signal is generated from tags or labels attached to signal or reporter agents or probes that have specific affinity to a target agent or analyte. Signal agents or probes may include, for example, signal antibodies or signal ligands, tagged with microspheres, sub-micron nanospheres, or enzymes. The microspheres or nanospheres may be fluorescent labeled (fluospheres), phosphorescent, luminecent, or chemiluminescent. The microspheres or nanospheres may also carry different chemical functionalities including, for example, carboxyl, amino, aldehyde, and hydrazine functional groups. These functional groups may facilitate binding of the signal agent. The enzyme may facilitate a chemical reaction that produces fluorescence, color, or a detectable signal in the presence of a suitable substrate. For example, conjugated horseradish peroxidase (HRP; Pierce, Rockford, Ill.) may be used with the substrate 3,3,5,5-tetramethylbenzidine (TMB; Calbiochem cat. no. 613548, CAS-54827-17-7) in the presence of hydrogen peroxide to produce an insoluble precipitate. Horseradish peroxidase can also be used in conjunction with CN/DAB (4-chloronaphthol/3,3′-diaminobenzidine, tetrahydrochloride), 4-CN (4-chloro-1-napthol), AEC (3-amino-9-ethyl carbazol) and DAB (3,3-diaminobenzidine tetrahydrochloride) to form insoluble precipitates. Similarly, the enzyme alkaline phosphatase (AP) can be used with the substrate bromochloroindolylphosphate in the practice of the present invention. Other suitable enzyme/substrate combinations will be apparent to those of skill in the art.

Detection

The signal from the microspheres or the enzyme reaction can be read with the optical bio-disc readers developed to be utilized in conjunction herewith. Either a bottom detector on a disc with a reflective cover, or a top detector with a transmissive disc may be employed as the optical bio-disc reader for the assay and disc systems and methods described herein.

Disc Implementation

In an advantageous embodiment, assays may be implemented on an analysis disc, modified optical disc, or bio-disc. The bio-disc may include a flow channel or fluidic circuit having one or more target or capture zones and/or bio-matrices embedded therein, a return channel in fluid communication therewith, a mixing chamber in fluid communication with the flow channel, and in some embodiments a waste reservoir in fluid communication with the flow channel.

The bio-disc may be implemented on an optical disc including an information encoding format such as CD, CD-R, or DVD or a modified version thereof. The bio-disc may include encoded information for performing, controlling, and post-processing the test or assay. For example, such encoded information may be directed to controlling the rotation rate of the disc, incubation time, incubation temperature, and/or specific steps of the assay. Depending on the test, assay, or investigational protocol, the rotation rate may be variable with intervening or consecutive sessions of acceleration, constant speed, and deceleration. These sessions may be closely controlled both as to speed and time of rotation to provide, for example, mixing, agitation, or separation of fluids and suspensions with agents, reagents, DNA, RNA, antigen, antibodies, ligands, and receptors.

Drive Implementation

A bio-disc drive assembly or reader may be employed to rotate the disc, read and process any encoded information stored on the disc, and analyze the samples in the flow channel of the bio-disc. The bio-disc drive is thus provided with a motor for rotating the bio-disc, a controller for controlling the rate of rotation of the disc, a processor for processing return signals from the disc, and an analyzer for analyzing the processed signals. The drive may include software specifically developed for performing the assays disclosed herein.

The rotation rate of the motor is controlled to achieve the desired rotation of the disc. The bio-disc drive assembly may also be utilized to write information to the bio-disc either before or after the test material in the flow channel and target or capture zone is interrogated by the read beam of the drive and analyzed by the analyzer. The bio-disc may include encoded information for controlling the rotation rate of the disc, providing processing information specific to the type of test to be conducted, and for displaying the results on a display monitor associated with the bio-drive in accordance with the assay methods relating hereto.

In one embodiment, an optical bio-disc includes a substrate having an inner perimeter and an outer perimeter; an operational layer associated with the substrate and including encoded information located along information tracks; and an analysis area including investigational features. The analysis area is positioned between the inner perimeter and the outer perimeter and is directed along the information tracks so that when an incident beam of electromagnetic energy tracks along them, the investigational features within the analysis area are thereby interrogated circumferentially. In another embodiment, the investigational features within the analysis area are interrogated according to a spiral path or, in general, according to a path of varying angular coordinate.

In one embodiment, the substrate includes a series of substantially circular information tracks that increase in circumference as a function of radius extending from the inner perimeter to the outer perimeter, the analysis area is circumferentially elongated between a pre-selected number of circular information tracks and the investigational features are interrogated substantially along the circular information tracks between a pre-selected inner and outer circumference.

In one embodiment, the analysis area includes a fluid chamber. Rotation of the bio-disc may be used to distribute investigational features in a substantially consistent distribution along the analysis area and/or in a substantially even distribution along the analysis area.

In another embodiment, the bio-disc includes a substrate having an inner perimeter and an outer perimeter; and an analysis zone including investigational features, the analysis zone being positioned between the inner perimeter and the outer perimeter of the substrate and extending according to a varying angular coordinate, and preferably according to a substantially circumferential or spiral path.

In one embodiment, the disc comprises an operational layer associated with the substrate and including encoded information located substantially along information tracks.

In another embodiment, the substrate includes a series of information tracks, of a substantially circular profile and increasing in circumference as a function of radius extending from the inner perimeter to the outer perimeter, and the analysis zone is directed substantially along the information tracks, so that when an incident beam of electromagnetic energy tracks along the information tracks, the investigational features within the analysis zone are thereby interrogated circumferentially. Alternatively, the analysis zone may be circumferentially elongated between a pre-selected number of circular information tracks, and the investigational features are interrogated substantially along the circular information tracks between a pre-selected inner and outer circumference.

In another embodiment, the analysis zone includes a plurality of reaction sites and/or a plurality of capture, analysis, or target zones arranged according to a varying angular coordinate. The optical analysis bio-disc may also include a plurality of analysis zones positioned between the inner perimeter and the outer perimeter of the substrate, at least one of which extends according to a varying angular coordinate.

In another embodiment, the disc includes multiple tiers of analysis zones, wherein each analysis zone extends according to a substantially circumferential path and each tier is arranged onto the bio-disc at a respective radial coordinate.

In a further preferred embodiment, the analysis zone includes one or more fluid chambers extending according to a varying angular coordinate, which chamber(s) has a central portion extending according to a varying angular coordinate and lateral arm portions extending according to a radial direction. In one embodiment, the chamber central portion has an angular extension θₐ being in a ratio θₐ/θ equal to or greater than 0.25 with the angle θ comprised between the chamber arm portions. Such embodiments may provide that the analysis zone includes at least a liquid-containing channel extending accordingly along a substantially circumferential path and the radius of curvature of the channel r_{c} and the length of the column of liquid b contained within the channel are in a ratio r_{c}/b equal to or greater than 0.5, and more preferably equal to or greater than 1.

In another embodiment, the optical analysis disc may include two inlet ports located at a lower radial coordinate of the bio-disc itself with respect to the analysis zone. Preferably, such ports are located each at one end of a respective lateral arm portion of the fluid chamber. Furthermore, the disc may include multiple tiers of analysis fluid channels, eventually comprising different assays, blood types, concentrations of cultured cells and the like. A set of fluid channels can also be arranged at substantially the same radial coordinate. Furthermore, the fluid channels can have the same or different sizes.

The disc may be either a reflective-type or transmissive-type optical bio-disc. As in previous embodiments, rotation of the bio-disc may be used to distribute investigational features in a substantially consistent and/or even distribution along the analysis zone.

In another embodiment, the optical analysis bio-disc may include a substrate having an inner perimeter and an outer perimeter; and an analysis zone including investigational features and positioned between the inner perimeter and the outer perimeter of the substrate. The analysis zone includes at least a liquid-containing channel having at least a portion which extends along a substantially circumferential path. The radius of curvature of the channel circumferential portion r_{c} and the length of the column of liquid b contained within the channel are preferably in a ratio r_{c}/b equal to or greater than 0.5. In another embodiment, the ratio r_{c}/b is equal to or greater than 1. Also in this embodiment, the disc can be either a reflective-type or a transmissive-type optical bio-disc.

In another embodiment, a method for the interrogation of investigational features within an optical analysis bio-disc provides interrogation of the investigational features according to a varying angular coordinate, and possibly according to a spiral or a substantially circumferential path. This interrogation step may also be such that when an incident beam of electromagnetic energy tracks along disc information tracks, investigational features within the analysis zone are thereby interrogated circumferentially. The interrogation step may provide interrogation of the investigational features according to a varying angular coordinate at a substantially fixed radial coordinate or, alternatively, according to a varying angular and radial coordinate. The interrogation step may provide interrogation of investigational features at a plurality of similar or different, reaction sites, capture zones, or target zones arranged according to a varying angular coordinate.

The above described methods and apparatus according to the present invention as disclosed herein can have one or more advantages which include, but are not limited to, simple and quick on-disc processing without the necessity of an experienced technician to run the test, small sample volumes, use of inexpensive materials, and use of known optical disc formats and drive manufacturing. These and other features and advantages will be better understood by reference to the following detailed description when taken in conjunction with the accompanying drawing figures and technical examples.

### BRIEF DESCRIPTION OF THE DRAWINGS

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1 is a pictorial representation of a bio-disc system;

FIG. 2 is an exploded perspective view of a reflective bio-disc; FIG. 3 is a top plan view of the disc shown in FIG. 2;

FIG. 4 is a perspective view of the disc illustrated in FIG. 3 with cut-away sections showing the different layers of the disc;

FIG. 5 is an exploded perspective view of a transmissive bio-disc;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 8 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17 is a pictorial graphical representation of the transformation of a sampled analog signal to a corresponding digital signal that is stored as a one-dimensional array;

FIG. 18 is a perspective view of an optical disc with an enlarged detailed view of an indicated section showing a captured white blood cell positioned relative to the tracks of the bio-disc yielding a signal-containing beam after interacting with an incident beam;

FIG. 19A is a graphical representation of a white blood cell positioned relative to the tracks of an optical bio-disc;

FIG. 19B is a series of signature traces derived from the white blood cell of FIG. 19A;

FIGS. 20A, 20B, 20C, and 20D, when taken together, form a pictorial graphical representation of transformation of the signature traces from FIG. 19B into digital signals that are stored as one-dimensional arrays and combined into a two-dimensional array for data input;

FIG. 21 is a logic flow chart depicting the principal steps for data evaluation according to processing methods and computational algorithms described herein;

FIGS. 22A, 22B, 22C, and 22D are cross-sectional side views of an optical bio-disc showing a method of detecting investigational features in a test sample.

FIGS. 23A, 23B, 23C, and 23D are cross-sectional side views of an optical bio-disc used in a mixed phase assay to detect investigational features in a test sample;

FIGS. 24A, 24B, 24C, 24D, 24E, and 24F are cross-sectional side views of an optical bio-disc showing a method of detecting investigational features in a test sample using ELISA;

FIG. 25 is a detailed partial cross-sectional view of the surface of a bio-disc showing reporter beads having specific affinity for antigens bound to the surface;

FIGS. 26A, 26B, 26C, and 26D are cross-sectional side views of an optical bio-disc showing a method of using reporter beads to detect investigational features in a test sample;

FIG. 27 is a detailed partial cross-sectional view of the surface of a bio-disc showing use of reporter beads, capture probes, and signal probes to detect in-vestigational features in a test sample;

FIG. 28 is view similar to FIG. 27, showing hybridization of the investigational feature to the capture and signal probes;

FIG. 29 is a cross-sectional side view of a bio-disc showing use of antibody-coated capture zones to detect analytes of interest in a test sample;

FIG. 30 is an exploded perspective view of an embodiment of bio-disc according to the present invention;

FIG. 31 is a top plan view of the disc of FIG. 30;

FIG. 32A is an exploded perspective view of a reflective bio-disc incorporating equi-radial channels of the present invention;

FIG. 32B is a top plan view of the disc shown in FIG. 32A;

FIG. 32C is a perspective view of the disc illustrated in FIG. 32B with cut-away sections showing the different layers of the e-radial reflective disc;

FIG. 33A is an exploded perspective view of a transmissive bio-disc utilizing the e-radial channels of the present invention;

FIG. 33B is a top plan view of the disc shown in FIG. 33A;

FIG. 33C is a perspective view of the disc illustrated in FIG. 33B with cut-away sections showing the different layers of this embodiment of the e-rad transmissive bio-disc;

FIGS. 34 and 35 are each a top plan views of a respective additional embodiment of the bio-disc of the present invention each shown in a bio-safe jewel case;

FIG. 36 is a pictorial representation of images derived from a transmissive optical bio-disc showing differences in signal derived from various concentrations of the hemoglobin;

FIG. 37 is a graphical representation of a dose response curve generated using the optical bio-disc system of the present invention;

FIG. 38 is a top plan view of another embodiment of the optical bio-disc having a micro-chromatographic matrix in a fluidic circuit;

FIG. 39A are top plan views of various layers of a chromatographic optical bio-disc of the present invention;

FIG. 39B is an exploded perspective view of the chromatographic optical bio-disc of FIG. 39A;

FIG. 39C is a partial cross sectional view taken perpendicular to a radius of the optical bio-disc illustrated in FIG. 39B showing the direction sample flow within the fluidic circuit;

FIG. 40A is an exploded perspective view of an alternative embodiment of the of the chromatographic optical bio-disc;

FIG. 40B is a top plan view of the optical bio-disc of FIG. 40A;

FIGS. 41A, 41B, and 41C shows steps for manufacturing the optical bio-disc for use in chromatographic assays;

FIGS. 42A and 42B shows steps for a method of using the optical bio-disc made according to the steps described in conjunction with FIGS. 41A-41C;

FIGS. 43A and 43B shows steps for a method of making the optical bio-disc for use in immuno-chemical and genetic assays;

FIGS. 44A, 44B, 44C, and 44D shows steps for a method of using the optical bio-disc made as illustrated in FIGS. 43A and 43B; and

FIG. 45 is a bar graph representation of results from a glycohemoglobin assay using the optical bio-disc.

### DETAILED DESCRIPTION OF THE PREFERRED EMBODIMENT

U.S. Pat. No. 6,030,581 entitled “Laboratory in a Disk” issued Feb. 29, 2000 (the '581 patent) is hereby incorporated by reference in its entirety. The '581 patent generally discloses an apparatus that includes an optical disc, adapted to be read by an optical reader, which has a sector having a substantially self-contained assay system useful for localizing and detecting an analyte suspected of being in a sample. U.S. Pat. No. 5,993,665, issued Nov. 30, 1999 (the '665 patent) entitled “Quantitative Cell Analysis Methods Employing Magnetic Separation” discloses analysis of biological specimens in a fluid medium where the specimens are rendered magnetically responsive by immuno-specific binding with ferromagnetic colloid. The '665 patent is hereby incorporated by reference in its entirety.

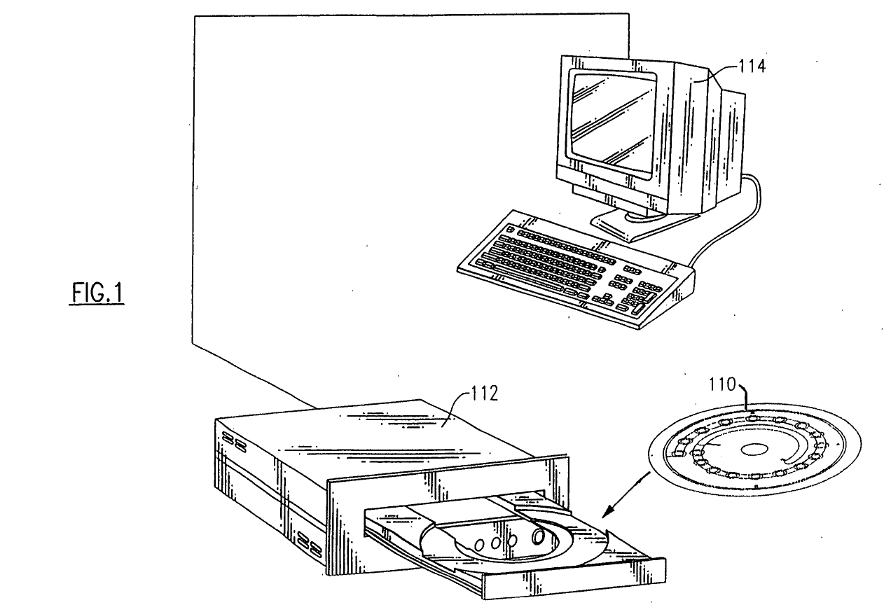

FIG. 1 is a perspective view of an optical bio-disc 110 for conducting biochemical analyses, and in particular cell counts and differential cell counts. The present optical bio-disc 110 is shown in conjunction with an optical disc drive 112 and a display monitor 114. Further details relating to this type of disc drive and disc analysis system are disclosed in U.S. patent application Ser. No. 10/008,156 entitled “Disc Drive System and Methods for Use with Bio-discs” filed Nov. 9, 2001 and U.S. patent application Ser. No. 10/043,688 entitled “Optical Disc Analysis System Including Related Methods For Biological and Medical Imaging” filed Jan. 10, 2002, both of which are herein incorporated by reference.

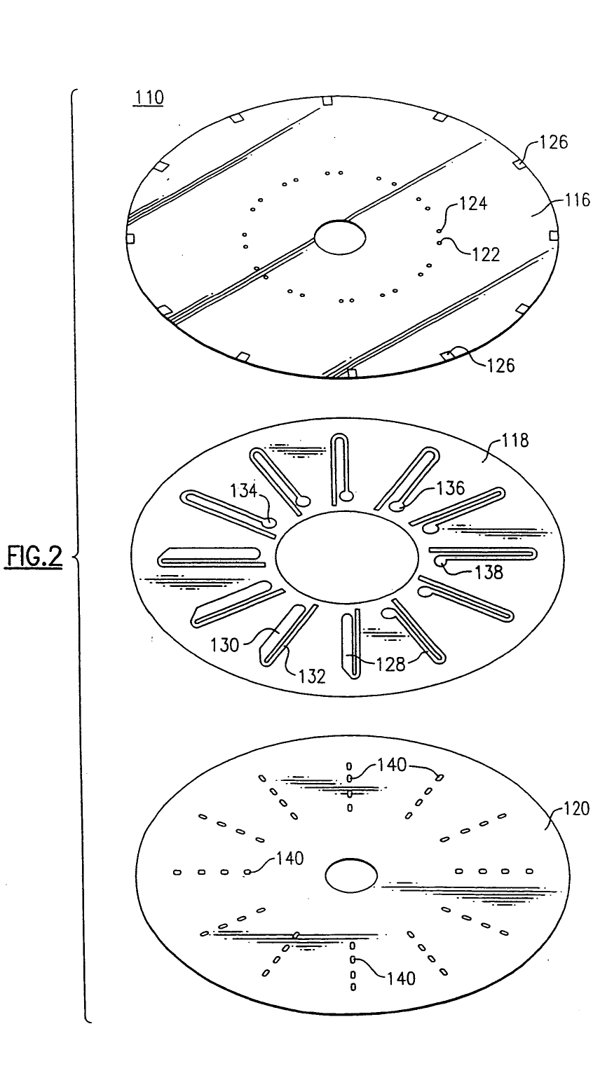

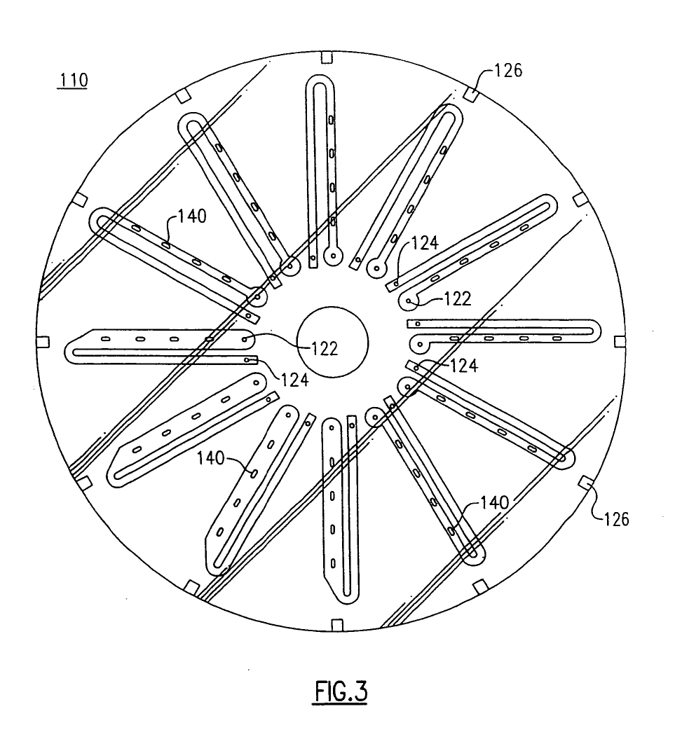

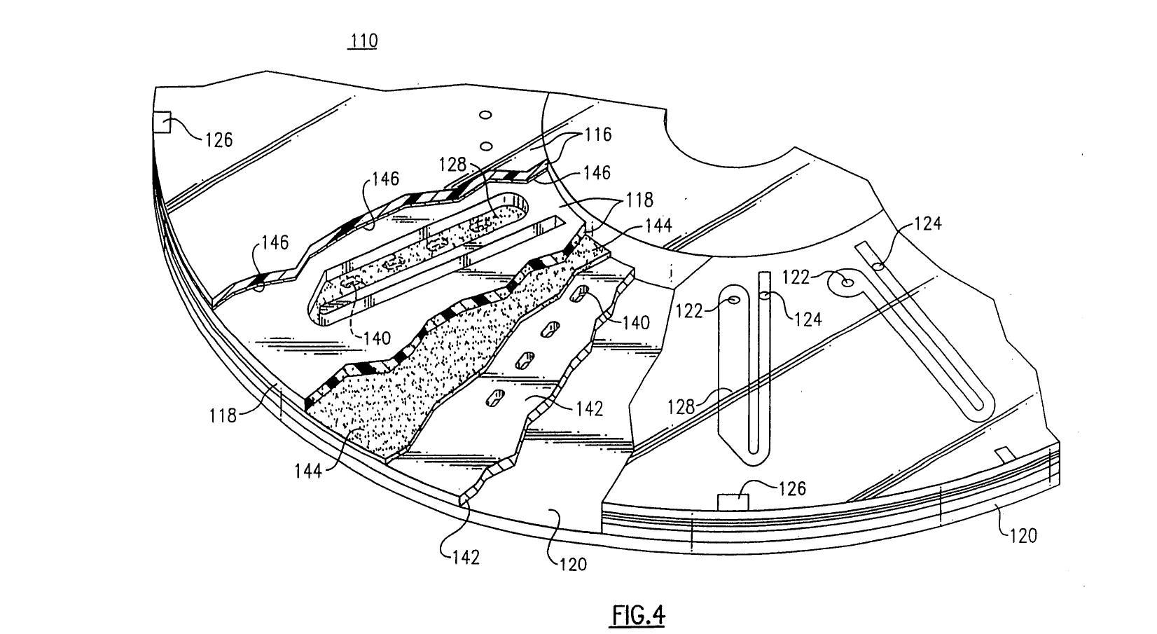

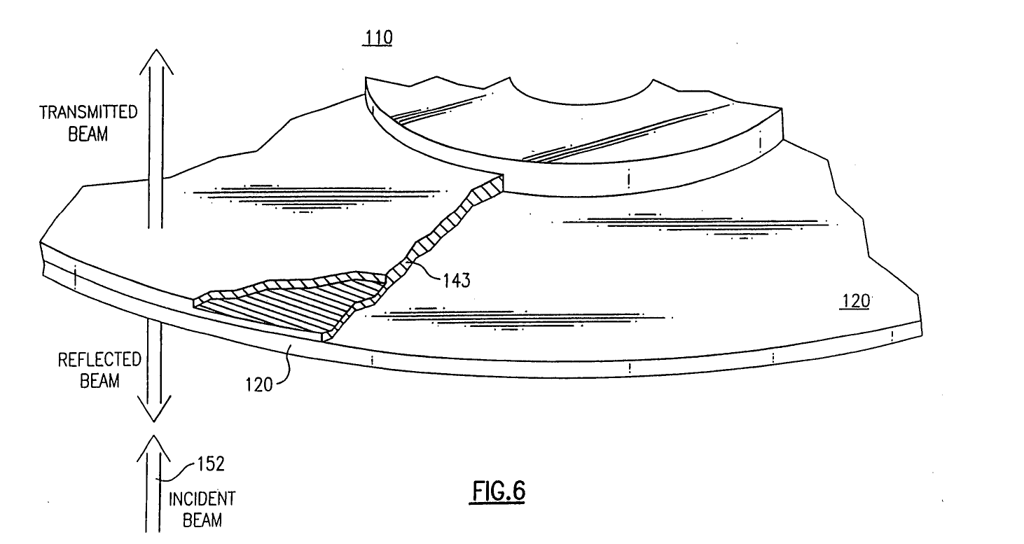

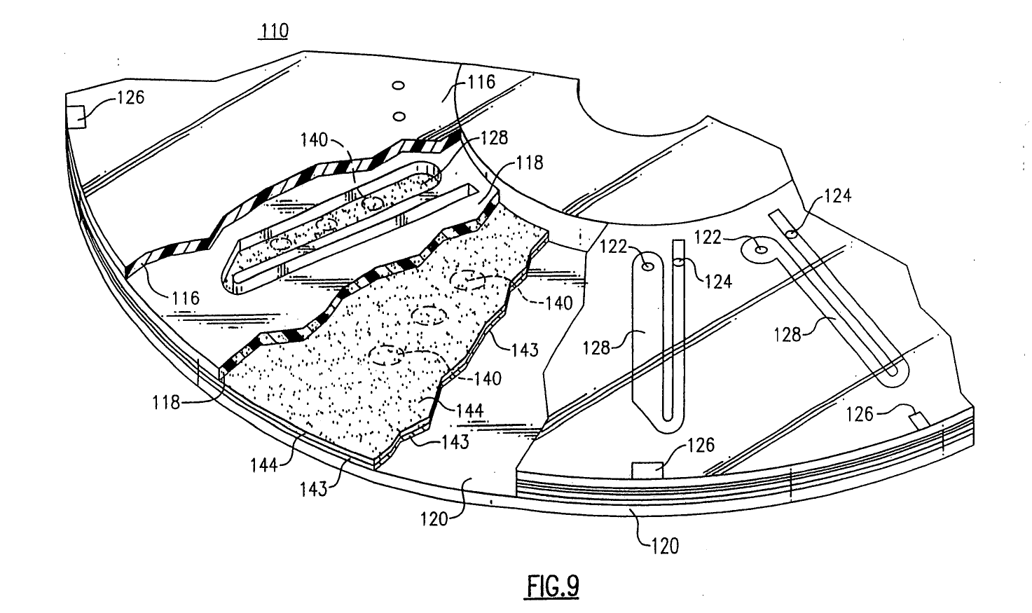

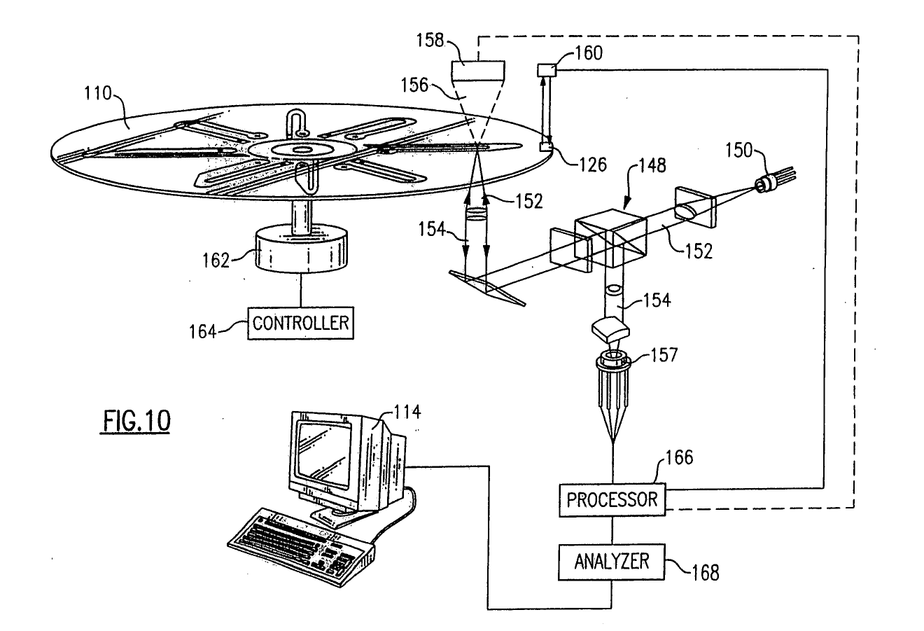

FIG. 2 is an exploded perspective view of the principal structural elements of one embodiment of the optical bio-disc 110. FIG. 2 is an example of a reflective zone optical bio-disc 110 (hereinafter “reflective disc”) that may be used according to the systems and methods described herein. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and may be coated with a reflective surface 146 (shown in FIG. 4) on the bottom thereof as viewed from the perspective of FIG. 2. In the preferred embodiment, trigger marks or markings 126 are included on the surface of a reflective layer 142 (shown FIG. 4). Trigger markings 126 may include a clear window in all three layers of the bio-disc, an opaque area, or a reflective or semi-reflective area encoded with information that sends data to a processor 166, as shown FIG. 10, that in turn interacts with the operative functions of an interrogation or incident beam 152, as shown in FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel or analysis zone 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has the aforementioned reflective layer 142 deposited on the top thereof (shown in FIG. 4). The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

FIG. 3 is a top plan view of the optical bio-disc 110 illustrated in FIG. 2 with the reflective layer 146 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment that may be used in the present invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In one embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively, as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

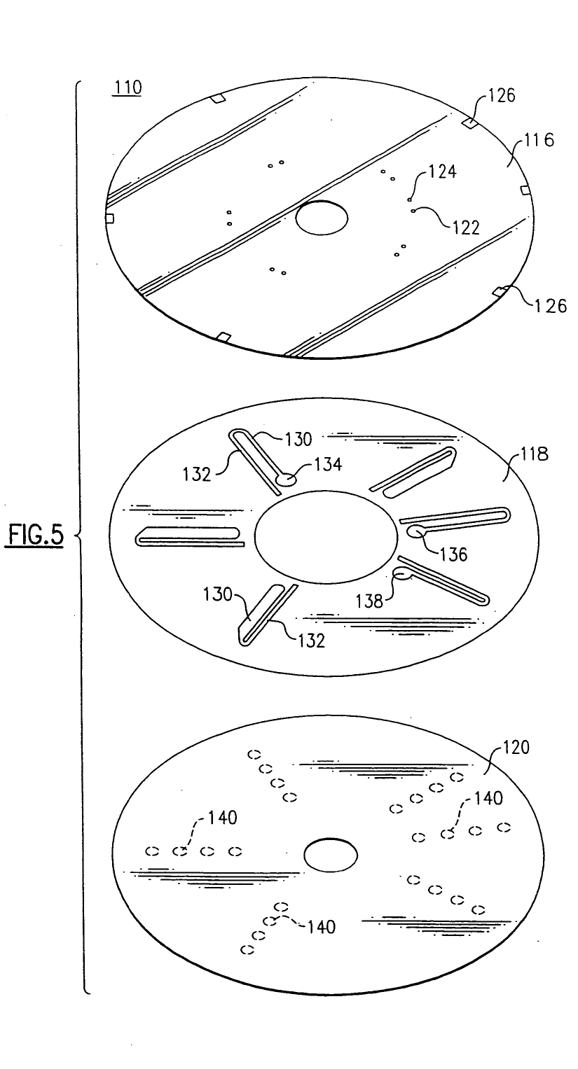

Referring now to FIG. 5, there is shown an exploded perspective view of the principal structural elements of a transmissive type of optical bio-disc 110. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 6 and 9. Trigger markings 126 may include a clear window in all three layers of the bio-disc, an opaque area, or a reflective or semi-reflective area encoded with information that sends data to a processor 166, FIG. 10, which in turn interacts with the operative functions of an interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 5 is the substrate 120 which may include target or capture zones 140. In one embodiment, the substrate 120 is made of polycarbonate and has the aforementioned thin semi-reflective layer 143 deposited on the top thereof, FIG. 6. The semi-reflective layer 143 associated with the substrate 120 of the disc 110 illustrated in FIGS. 5 and 6 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi- reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158. FIGS. 10 and 12, while some of the light is reflected or returned back along the incident path. As indicated below, Table 1 presents the reflective and transmissive characteristics of a gold film relative to the thickness of the film. The gold film layer is fully reflective at a thickness greater than 800 Å. While the threshold density for transmission of light through the gold film is approximately 400 Å.

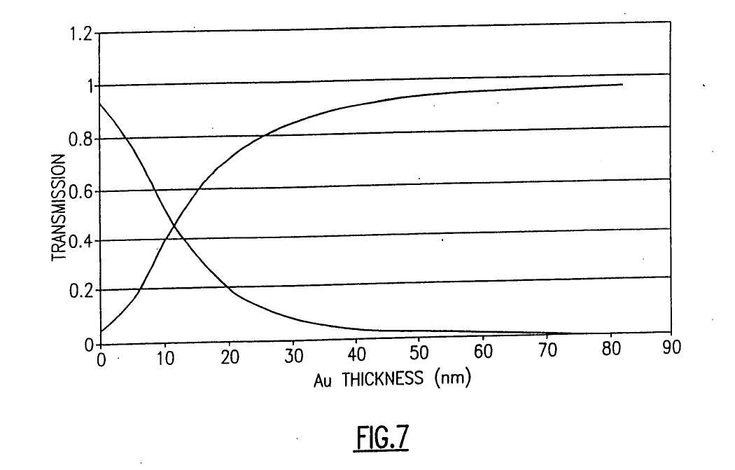

In addition to Table 1, FIG. 7 provides a graphical representation of the inverse relationship of the reflective and transmissive nature of the thin semi-reflective layer 143 based upon the thickness of the gold. Reflective and transmissive values used in the graph illustrated in FIG. 7 are absolute values.

TABLE 1
Au film Reflection and Transmission (Absolute Values)

| Thickness (Angstroms) | Thickness (nm) | Reflectance | Transmittance |
|---|---|---|---|
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

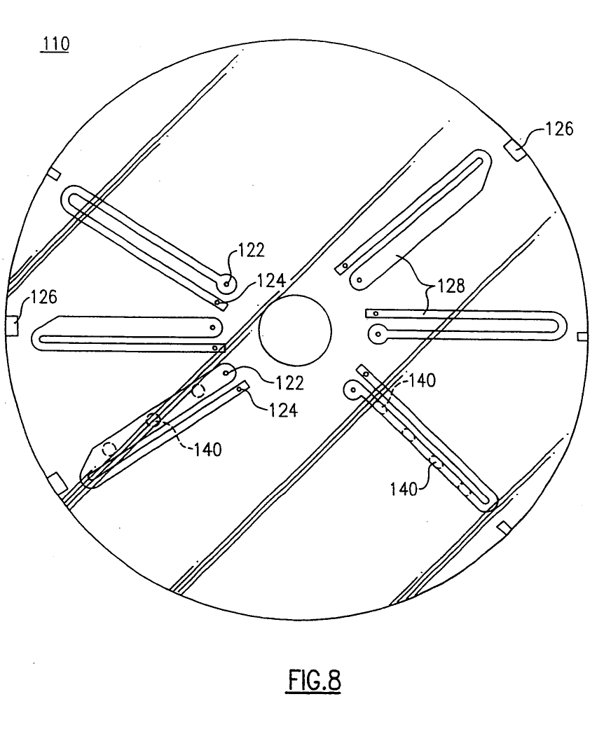

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment. The disc 110 is illustrated with a portion of the various layers thereof cut away to show a partial sectional view of each principal layer, substrate, coating, or membrane. FIG. 9 illustrates a transmissive disc format with the clear cap portion 116, the think semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from a trigger detector 160, FIG. 10. FIG. 9 also shows the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by the aforementioned top detector 158, and is also analyzed for the presence of signal elements. In the transmissive embodiment, a photo detector may be used as top detector 158.

FIG. 10 also shows a hardware trigger mechanism that includes the trigger markings 126 on the disc and the aforementioned trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140, e.g. at a predetermined reaction site. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated with the transmissive optical bio-disc.

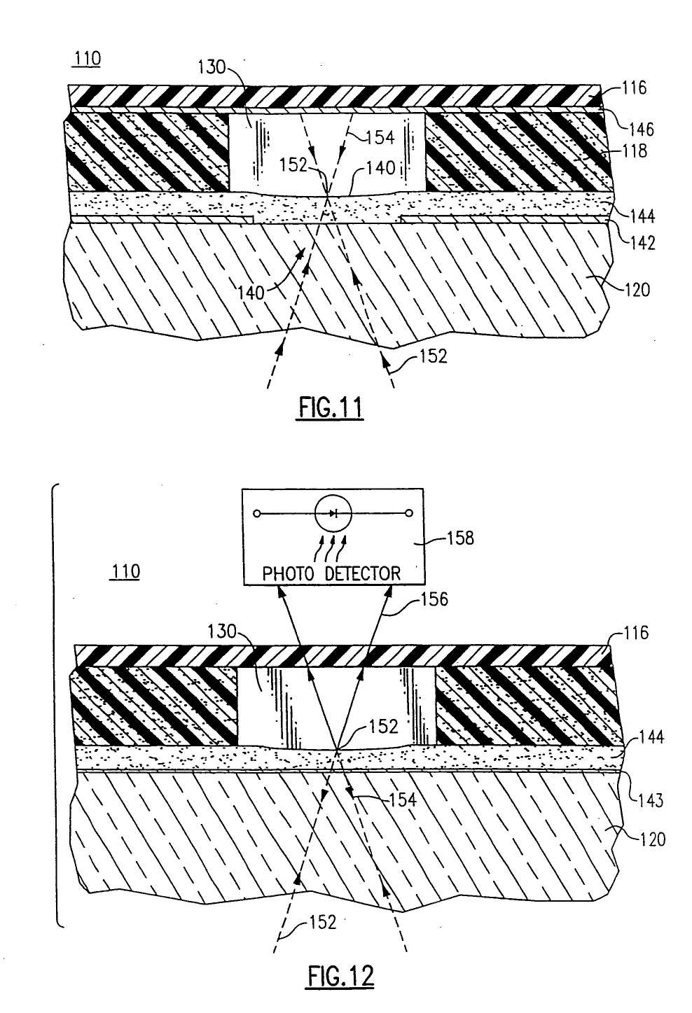

As shown in FIG. 11, there is presented a partial cross sectional view of the reflective disc embodiment of the optical bio-disc 110. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

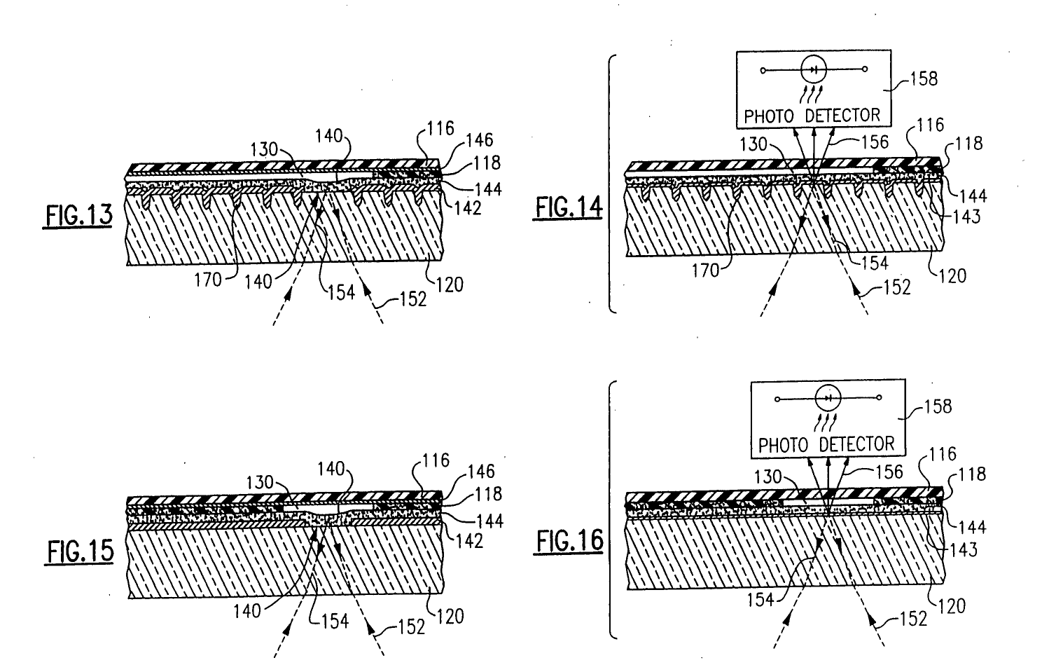

FIG. 12 is a partial cross sectional view of the transmissive embodiment of the bio-disc 110. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

Counting Methods and Related Software

By way of illustrative background, a number of methods and related algorithms for white blood cell counting using optical disc data are herein discussed in further detail. These methods and related algorithms are not limited to counting white blood cells, but may be readily applied to conducting counts of any type of particulate matter including, but not limited to, red blood cells, white blood cells, beads, and any other objects, both biological and non-biological, that produce similar optical signatures that can be detected by an optical reader.

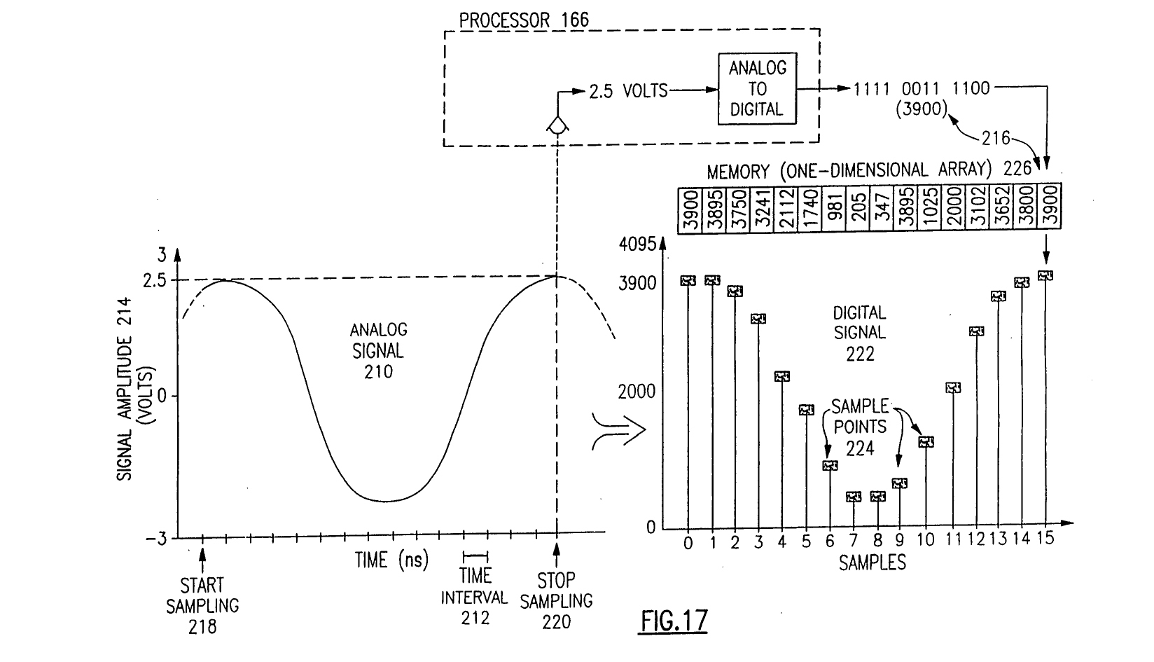

For the purposes of illustration, the following description of the methods and algorithms related to the present invention as described with reference to FIGS. 17-21, are directed to cell counting. With some modifications, these methods and algorithms can be applied to counting other types of objects. The data evaluation aspects of the cell counting methods and algorithms are described generally herein to provide related background for the methods and apparatus of the present invention. In the following discussion, the basic scheme of the methods and algorithms with a brief explanation is presented. As illustrated in FIG. 10, information concerning attributes of the biological test sample is retrieved from the optical bio-disc 110 in the form of a beam of electromagnetic radiation that has been modified or modulated by interaction with the test sample. In the case of the reflective optical bio-disc discussed in conjunction with FIGS. 2, 3, 4, 11, 13, and 15, the return beam 154 carries the information about the biological sample. As discussed above, such information about the biological sample is contained in the return beam essentially only when the incident beam is within the flow channel 130 or target zones 140 and thus in contact with the sample. In the reflective embodiment of the bio-disc 110, the return beam 154 may also carry information encoded in or on the reflective layer 142 or otherwise encoded in the wobble grooves 170 illustrated in FIGS. 13 and 14. As would be apparent to one of skill in the art, pre-recorded information is contained in the return beam 154 of the reflective disc with target zones, only when the corresponding incident beam is in contact with the reflective layer 142. Such information is not contained in the return beam 154 when the incident beam 152 is in an area where the information bearing reflective layer 142 has been removed or is otherwise absent. In the case of the transmissive optical bio-disc discussed in conjunction with FIGS. 5, 6, 8, 9, 12, 14, and 16, the transmitted beam 156 carries the information about the biological sample.

With continuing reference to FIG. 10, the information about the biological test sample, whether it is obtained from the return beam 154 of the reflective disc or the transmitted beam 156 of the transmissive disc, is directed to processor 166 for signal processing. This processing involves transformation of the analog signal detected by the bottom detector 157 (reflective disc) or the top detector 158 (transmissive disc) to a discrete digital form.

Referring next to FIG. 17, the signal transformation involves sampling the analog signal 210 at fixed time intervals 212, and encoding the corresponding instantaneous analog amplitude 214 of the signal as a discrete binary integer 216. Sampling is started at some start time 218 and stopped at some end time 220. The two common values associated with any analog-to-digital conversion process are sampling frequency and bit depth. The sampling frequency, also called the sampling rate, is the number of samples taken per unit time. A higher sampling frequency yields a smaller time interval 212 between consecutive samples, which results in a higher fidelity of the digital signal 222 compared to the original analog signal 210. Bit depth is the number of bits used in each sample point to encode the sampled amplitude 214 of the analog signal 210. The greater the bit depth, the better the binary integer 216 will approximate the original analog amplitude 214. In the present embodiment, the sampling rate is 8 MHz with a bit depth of 12 bits per sample, allowing an integer sample range of 0 to 4095 (0 to (2n−1), where n is the bit depth. This combination may change to accommodate the particular accuracy necessary in other embodiments. By way of example and not limitation, it may be desirable to increase sampling frequency in embodiments involving methods for counting beads, which are generally smaller than cells. The sampled data is then sent to processor 166 for analog-to-digital transformation.

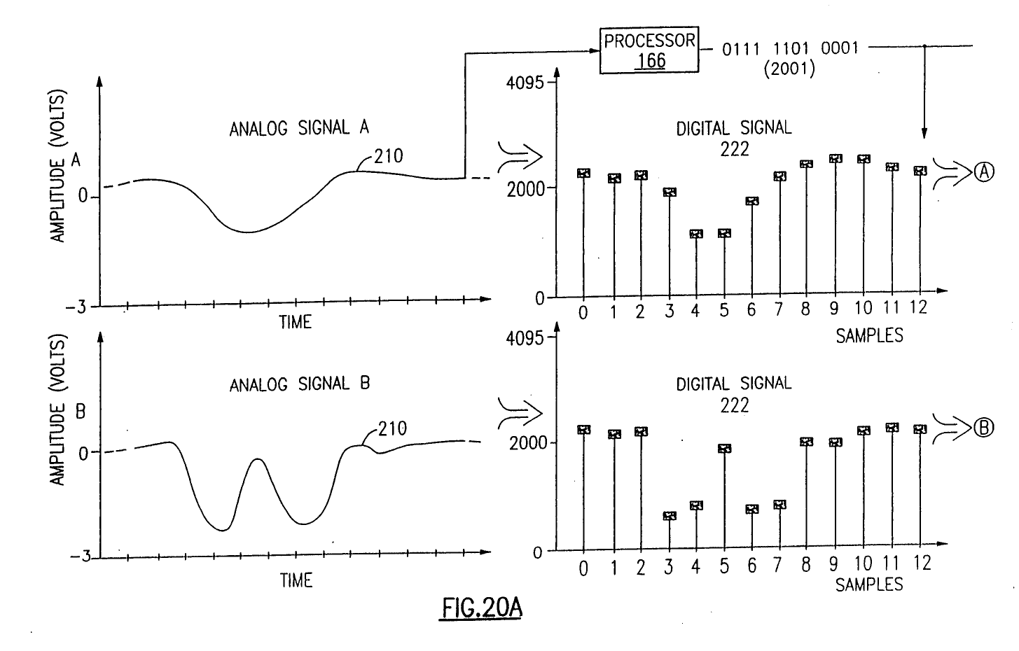

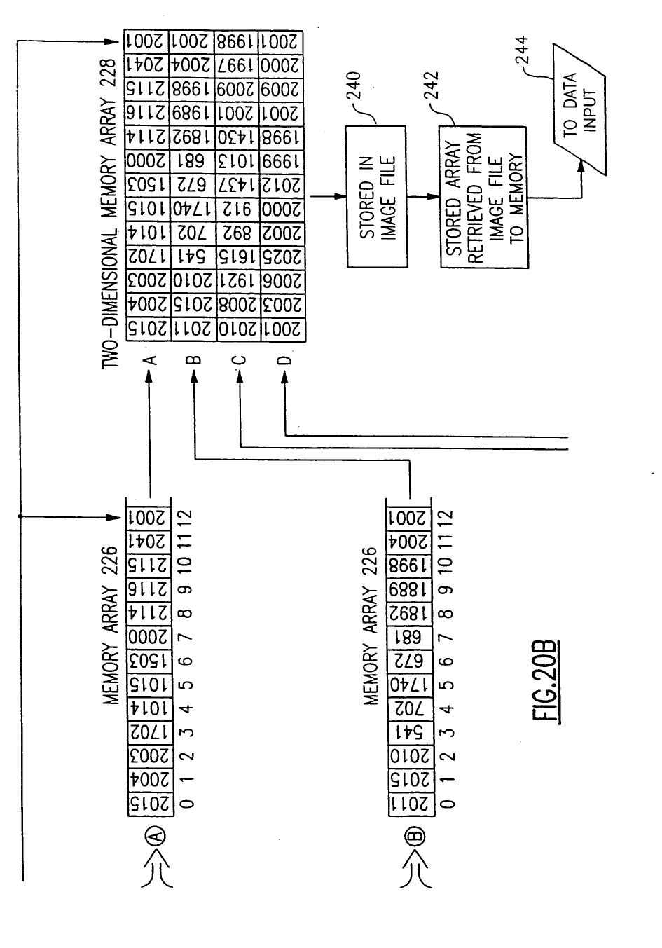

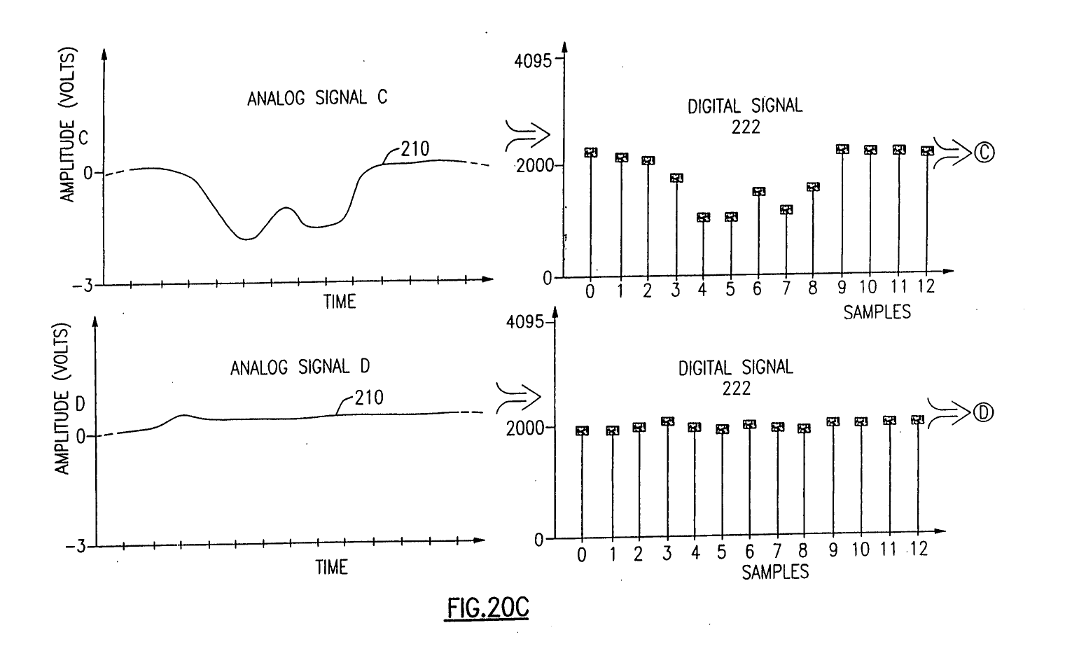

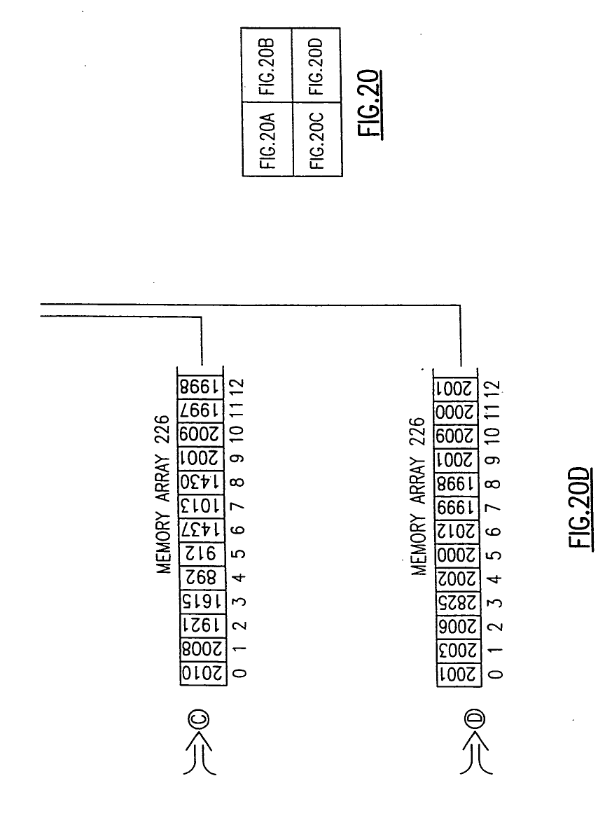

During the analog-to-digital transformation, each consecutive sample point 224 along the laser path is stored consecutively on disc or in memory as a one-dimensional array 226. Each consecutive track contributes an independent one-dimensional array, which yields a two-dimensional array 228 (FIG. 20A) that is analogous to an image.

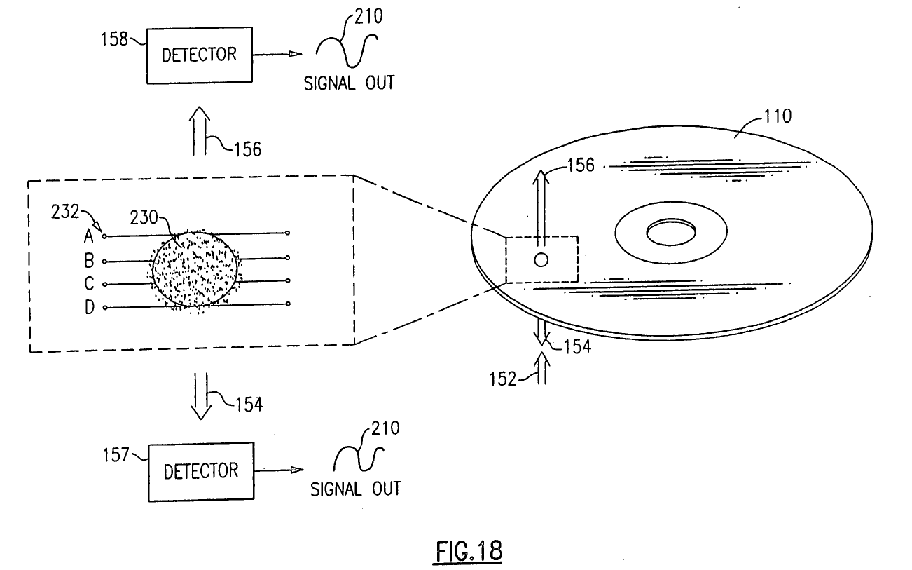

FIG. 18 is a perspective view of an optical bio-disc 110 with an enlarged detailed perspective view of the section indicated showing a captured white blood cell 230 positioned relative to the tracks 232 of the optical bio-disc. The white blood cell 230 is used herein for illustrative purposes only. As indicated above, other objects or investigational features such as beads or agglutinated matter may be utilized herewith. As shown, the interaction of incident beam 152 with white blood cell 230 yields a signal-containing beam, either in the form of a return beam 154 of the reflective disc or a transmitted beam 156 of the transmissive disc, which is detected by either of detectors 157 or 158.

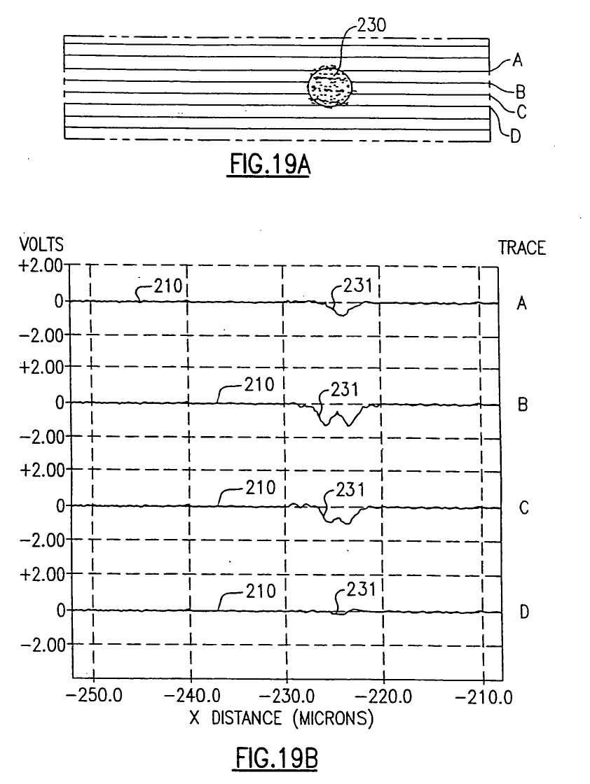

FIG. 19A is another graphical representation of the white blood cell 230 positioned relative to the tracks 232 of the optical bio-disc 110 shown in FIG. 18. As shown in FIGS. 18 and 19A, the white blood cell 230 covers approximately four tracks A, B, C, and D. FIG. 19B shows a series of signature traces derived from the white blood cell 210 of FIGS. 19 and 19A. As indicated in FIG. 19B, the detection system provides four analogue signals A, B, C, and D corresponding to tracks A, B, C, and D. As further shown in FIG. 19B, each of the analogue signals A, B, C, and D carries specific information about the white blood cell 230. Thus as illustrated, a scan over a white blood cell 230 yields distinct perturbations of the incident beam that can be detected and processed. The analog signature traces (signals) 210 are then directed to processor 166 for transformation to an analogous digital signal 222 as shown in FIGS. 20A and 20C as discussed in further detail below.

FIG. 20 is a graphical representation illustrating the relationship between FIGS. 20A, 20B, 20C, and 20D. FIGS. 20A, 20B, 20C, and 20D are pictorial graphical representations of transformation of the signature traces from FIG. 19B into digital signals 222 that are stored as one-dimensional arrays 226 and combined into a two-dimensional array 228 for data input 244.

With particular reference now to FIG. 20A, there is shown sampled analog signals 210 from tracks A and B of the optical bio-disc shown in FIGS. 18 and 19A. Processor 166 then encodes the corresponding instantaneous analog amplitude 214 of the analog signal 210 as a discrete binary integer 216 (see FIG. 17). The resulting series of data points is the digital signal 222 that is analogous to the sampled analog signal 210.

Referring next to FIG. 20B, digital signal 222 from tracks A and B (FIG. 20A) is stored as an independent one-dimensional memory array 226. Each consecutive track contributes a corresponding one-dimensional array, which when combined with the previous one-dimensional arrays, yields a two-dimensional array 228 that is analogous to an image. The digital data is then stored in memory or on disc as a two-dimensional array 228 of sample points 224 (FIG. 17) that represent the relative intensity of the return beam 154 or transmitted beam 156 (FIG. 18) at a particular point in the sample area. The two-dimensional array is then stored in memory or on disc in the form of a raw file or image file 240 as represented in FIG. 20B. The data stored in the image file 240 is then retrieved 242 to memory and used as data input 244 to analyzer 168 shown in FIG. 10.

FIG. 20C shows sampled analog signals 210 from tracks C and D of the optical bio-disc shown in FIGS. 18 and 19A. Processor 166 then encodes the corresponding instantaneous analog amplitude 214 of the analog signal 210 as a discrete binary integer 216 (FIG. 17). The resulting series of data points is the digital signal 222 that is analogous to the sampled analog signal 210.

Referring now to FIG. 20D, digital signal 222 from tracks C and D is stored as an independent one-dimensional memory array 226. Each consecutive track contributes a corresponding one-dimensional array, which when combined with the previous one-dimensional arrays, yields a two-dimensional array 228 that is analogous to an image. As above, the digital data is then stored in memory or on disc as a two-dimensional array 228 of sample points 224 (FIG. 17) that represent the relative intensity of the return beam 154 or transmitted beam 156 (FIG. 18) at a particular point in the sample area. The two-dimensional array is then stored in memory or on disc in the form of a raw file or image file 240 as shown in FIG. 20B. As indicated above, the data stored in the image file 240 is then retrieved 242 to memory and used as data input 244 to analyzer 168 FIG. 10.

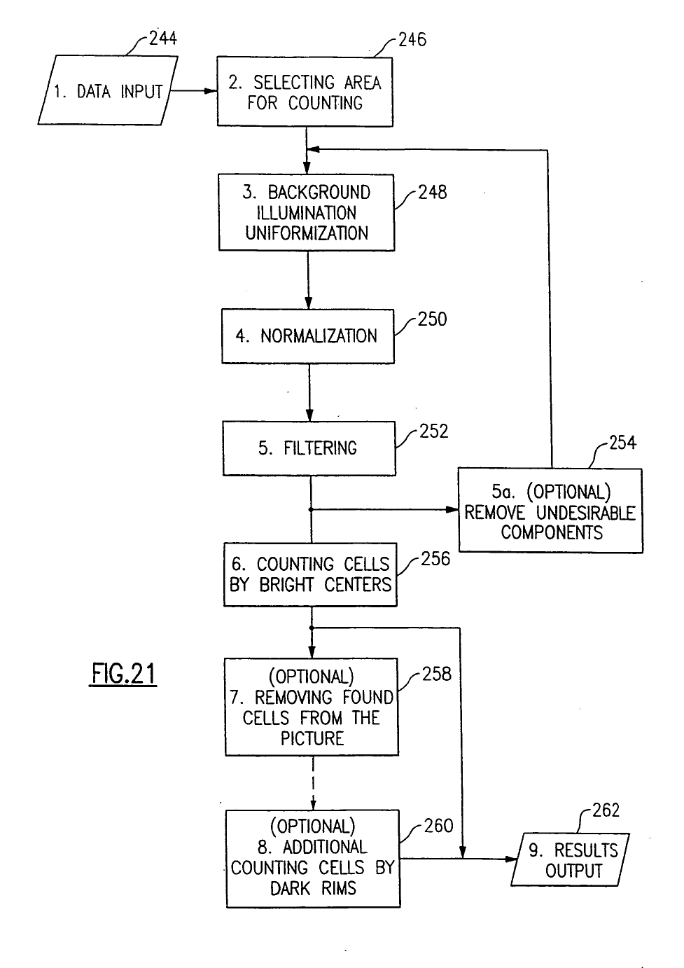

The computational and processing algorithms are stored in analyzer 168 (FIG. 10) and applied to the input data 244 to produce useful output results 262 (FIG. 21) that may be displayed on the display monitor 114 (FIG. 10).

With reference now to FIG. 21 there is shown a logic flow chart of the principal steps for data evaluation according to the processing methods and computational algorithms related to the present invention. A first principal step of the present processing method involves receipt of the input data 244. As described above, data evaluation starts with an array of integers in the range of 0 to 4096.

The next principle step 246 is selecting an area of the disc for counting. Once this area is defined, an objective then becomes making an actual count of all white blood cells contained in the defined area. The implementation of step 246 depends on the configuration of the disc and user's options. By way of example and not limitation, embodiments of the invention using discs with windows such as the target zones 140 shown in FIGS. 2 and 5, the software recognizes the windows and crops a section thereof for analysis and counting. In one preferred embodiment, such as that illustrated in FIG. 2, the target zones or windows have the shape of 1×2 mm rectangles with a semicircular section on each end thereof. In this embodiment, the software crops a standard rectangle of 1×2 mm area inside a respective window. In an aspect of this embodiment, the reader may take several consecutive sample values to compare the number of cells in several different windows.

In embodiments of the invention using a transmissive disc without windows, as shown in FIGS. 5, 6, 8, and 9, step 246 may be performed in one of two different manners. The position of the standard rectangle is chosen either by positioning its center relative to a point with fixed coordinates, or by finding reference mark which may be a spot of dark dye. In the case where a reference mark is employed, a dye with a desired contrast is deposited in a specific position on the disc with respect to two clusters of cells. The optical disc reader is then directed to skip to the center of one of the clusters of cells and the standard rectangle is then centered around the selected cluster.

As for the user options mentioned above in regard to step 246, the user may specify a desired sample area shape for cell counting, such as a rectangular area, by direct interaction with mouse selection or otherwise. In the present embodiment of the software, this involves using the mouse to click and drag a rectangle over the desired portion of the optical bio-disc-derived image that is displayed on monitor 114. Regardless of the evaluation area selection method, a respective rectangular area is evaluated for counting in the next step 248.

The third principal step in FIG. 21 is step 248, which is directed to background illumination uniformization. This process corrects possible background uniformity fluctuations caused in some hardware configurations. Background illumination uniformization offsets the intensity level of each sample point such that the overall background, or the portion of the image that is not cells, approaches a plane with an arbitrary background value Vbackground. While Vbackground may be decided in many ways, such as taking the average value over the standard rectangular sample area, in the present embodiment, the value is set to 2000. The value V at each point P of the selected rectangular sample area is replaced with the number (Vbackground+(V−average value over the neighborhood of P)) and truncated, if necessary, to fit the actual possible range of values, which is 0 to 4095 in a preferred embodiment of the present invention. The dimensions of the neighborhood are chosen to be sufficiently larger than the size of a cell and sufficiently smaller than the size of the standard rectangle.

The next step in the flow chart of FIG. 21 is a normalization step 250. In conducting normalization step 250, a linear transform is performed with the data in the standard rectangular sample area so that the average becomes 2000 with a standard deviation of 600. If necessary, the values are truncated to fit the range 0 to 4096. This step 250, as well as the background illumination uniformization step 248, makes the software less sensitive to hardware modifications and tuning. By way of example and not limitation, the signal gain in the detection circuitry, such as top detector 158 (FIG. 18), may change without significantly affecting the resultant cell counts.

As shown in FIG. 21, a filtering step 252 is next performed. For each point P in the standard rectangle, the number of points in the neighborhood of P, with dimensions smaller than indicated in step 248, with values sufficiently distinct from Vbackground is calculated. The points calculated should approximate the size of a cell in the image. If this number is large enough, the value at P remains as it was; otherwise it is assigned to Vbackground. This filtering operation is performed to remove noise, and in the optimal case only cells remain in the image while the background is uniformly equal Vbackground.

An optional step 254 directed to removing bad components may be performed as indicated in FIG. 21. Defects such as scratches, bubbles, dirt, and other similar irregularities may pass through filtering step 252. These defects may cause cell counting errors either directly or by affecting the overall distribution in the images histogram. Typically, these defects are sufficiently larger in size than cells and can be removed in step 254 as follows. First a binary image with the same dimensions as the selected region is formed. A in the binary image is defined as white, if the value at the corresponding point of the original image is equal to Vbackground, and black otherwise. Next, connected components of black points are extracted. Then subsequent erosion and expansion are applied to regularize the view of components. And finally, components that are larger than a defined threshold are removed. In one embodiment of this optional step, the component is removed from the original image by assigning the corresponding sample points in the original image with the value Vbackground. The threshold that determines which components constitute countable objects and which are to be removed is a user-defined value. This threshold may also vary depending on the investigational feature being counted i.e. white blood cells, red blood cells, or other biological matter. After optional step 254, steps 248, 250, and 252 are preferably repeated.

The next principal processing step shown in FIG. 21 is step 256, which is directed to counting cells by bright centers. The counting step 256 consists of several substeps. The first of these substeps includes performing a convolution. In this convolution substep, an auxiliary array referred to as a convolved picture is formed. The value of the convolved picture at point P is the result of integration of a picture after filtering in the circular neighborhood of P. More precisely, for one specific embodiment, the function that is integrated, is the function that equals v-2000 when v is greater than 2000 and 0 when v is less than or equal to 2000. The next substep performed in counting step 256 is finding the local maxima of the convolved picture in the neighborhood of a radius about the size of a cell. Next, duplicate local maxima with the same value in a closed neighborhood of each other are avoided. In the last substep in counting step 256, the remaining local maxima are declared to mark cells.

In some hardware configurations, some cells may appear without bright centers. In these instances, only a dark rim is visible and the following two optional steps 258 and 260 are useful.

Step 258 is directed to removing found cells from the picture. In step 258, the circular region around the center of each found cell is filled with the value 2000 so that the cells with both bright centers and dark rims would not be found twice.

Step 260 is directed to counting additional cells by dark rims. Two transforms are made with the image after step 258. In the first substep of this routine, substep (a), the value v at each point is replaced with (2000-v) and if the result is negative it is replaced with zero. In substep (b), the resulting picture is then convolved with a ring of inner radius R1 and outer radius R2. R1 and R2 are, respectively, the minimal and the maximal expected radius of a cell, the ring being shifted, subsequently, in substep (d) to the left, right, up and down. In substep (c), the results of four shifts are summed. After this transform, the image of a dark rim cell looks like a four petal flower. Finally in substep (d), maxima of the function obtained in substep (c) are found in a manner to that employed in counting step 256. They are declared to mark cells omitted in step 256.

After counting step 256, or after counting step 260 when optionally employed, the last principal step illustrated in FIG. 21 is a results output step 262. The number of cells found in the standard rectangle is displayed on the monitor 114 shown in FIGS. 1 and 5, and each cell identified is marked with a cross on the displayed optical bio-disc-derived image.

On-Disc Biological and Chemical Assays

The following discussion is directed to the biological and chemical applications for which the invention is useful. In sequencing applications, a sequence of nucleotide bases within the DNA sample can be determined by detecting which probes have the DNA sample bound thereto. In diagnostic applications, a genomic sample from an individual is screened against a predetermined set of probes to determine if the individual has a disease or a genetic disposition to a disease.

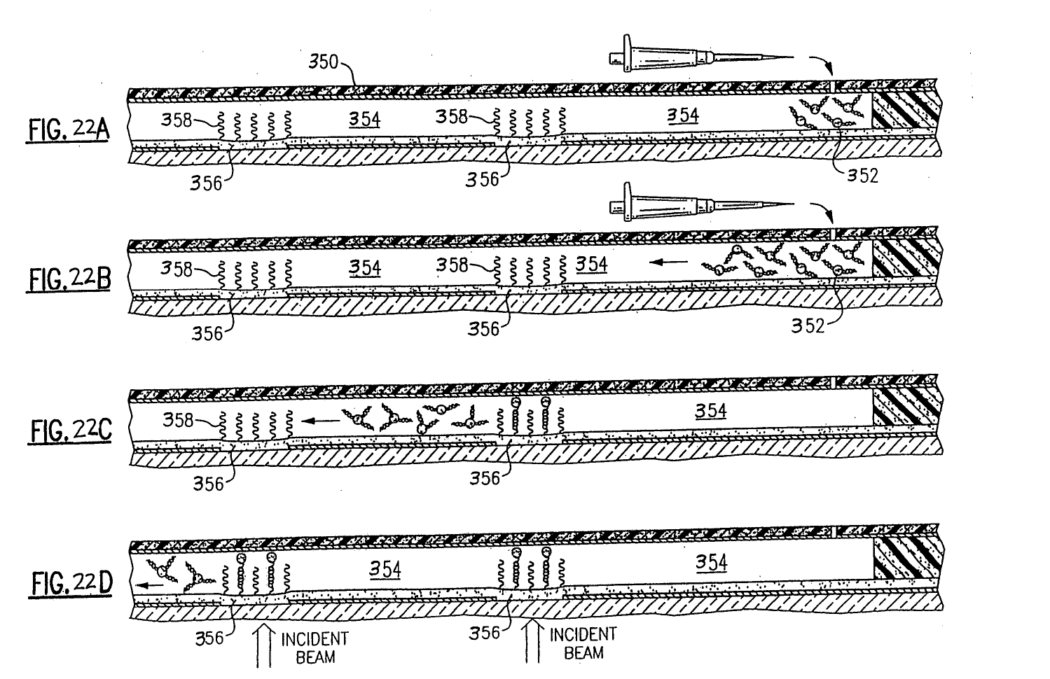

This invention combines microfluidic technology with genomics and proteomics on an optical bio-disc to detect investigational features in a test sample. Referring to FIGS. 22A, 22B, 22C, and 22D, an aqueous test sample 352 is placed on or within an optical bio-disc 350 and is driven through micro-channels 354 across a specially prepared surface 356 to effectuate the desired tests. Capillary action, pressure applied with an external applicator, and/or centrifugal force (i.e., the force on a body in curvilinear motion directed away from the center or curvature or axis of rotation) act upon the test sample to achieve contact with capture probes 358. Nucleic acid probe technology has application in detection of genetic mutations and related mechanisms, cancer screening, determining drug toxicity levels, detection of genetic disorders, detection of infectious disease, and genetic fingerprinting.

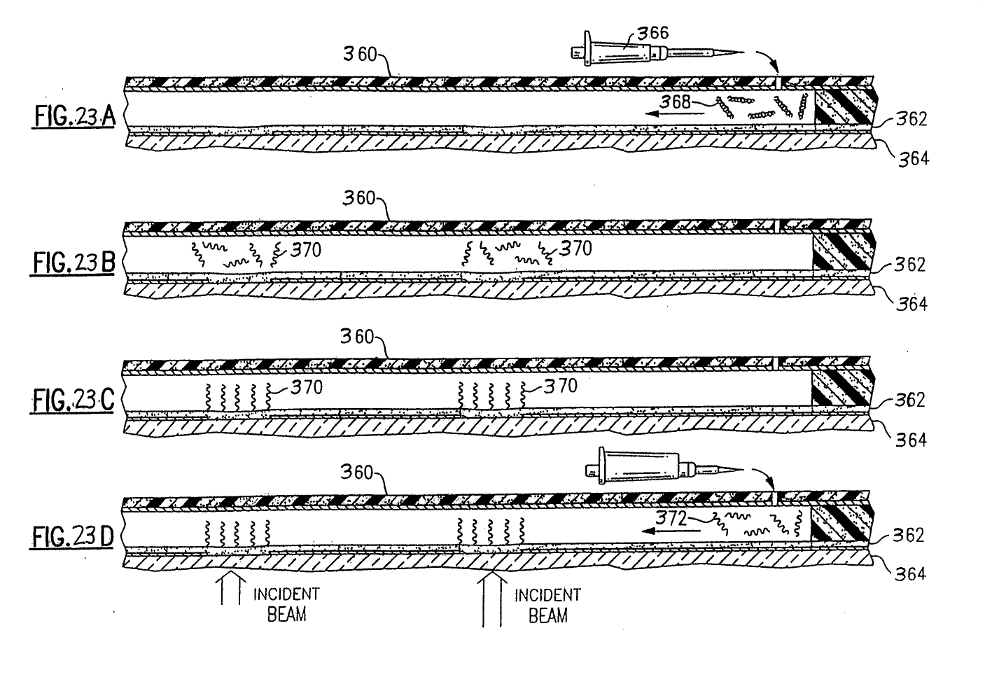

Additionally, the invention is adapted for use in a mixed phase system to perform hybridization assays. Referring to FIGS. 23A, 23B, 23C, and 23D, a mixed phase assay involves performing hybridizations on a solid phase such as a thin nylon or nitrocellulose membrane 362. For example, the assays usually involve spin-coating a thin layer of nitrocellulose 362 onto the substrate 364 of a bio-disc 360, using a pipette 366 or similar device to load the membrane with a sample 368, denaturing the DNA or creating single stranded molecules 370, fixing the DNA or RNA to the membrane, and saturating the remaining membrane attachment sites with heterologous nucleic acids and/or proteins 372 to prevent the analytes and reporters from adhering to the membrane in a non-specific manner. All of these steps must be carried out before performing the actual hybridization. Subsequent steps are then performed to achieve hybridization and locate reporter beads in the capture areas or target zones. The incident beam is then utilized to detect the reporters as discussed in reference to FIG. 22.

Optical bio-discs are useful for experimental analysis and assays in the areas of genetics and proteomics in applications as diverse as pharmaco-genomics, gene expression, compound screening, toxicology, forensic investigation, Single Nucleotide Polymorphism (SNPs) analysis, Short Tandem Repeats (STRs), and clinical/molecular diagnostics.

Reporters

Many chemical, biochemical, and biological assays rely upon inducing a change in the optical properties of the particular sample being tested. Such a change may occur upon detection of the investigational feature itself (e.g., blood cells), or upon detection of a reporter. In the case where investigational features are too small to be detected by the read beam of the optical disc drive, reporters having a selective affinity (i.e., a tendency to react or combine with atoms or compounds of different chemical constitution for the investigational features within the test sample) for the investigational feature to facilitate detection. The reporter will react, combine, or otherwise bind to the investigational feature, thereby causing a detectable color, chemiluminescent, luminescent, or other identifiable label into the investigational feature.

Luminescence is formally divided into two categories, fluorescence and phosphorescence, depending on the nature of the excited state. A luminescent molecule has the ability to absorb photons of energy at one wavelength and subsequently emit the energy at another wavelength. Luminescence is caused by incident radiation impinging upon or exciting an electron of a molecule. The electron absorbs the incident radiation and is raised from a lower quantum energy level to a higher one. The excess energy is released as photons of light as the electron returns to the lower, ground-state energy level. Since each reporter has its own luminescent character, more than one labeled molecule, each tagged with a different reporter, can be used at the same time to detect two or more investigational features within the same test sample.

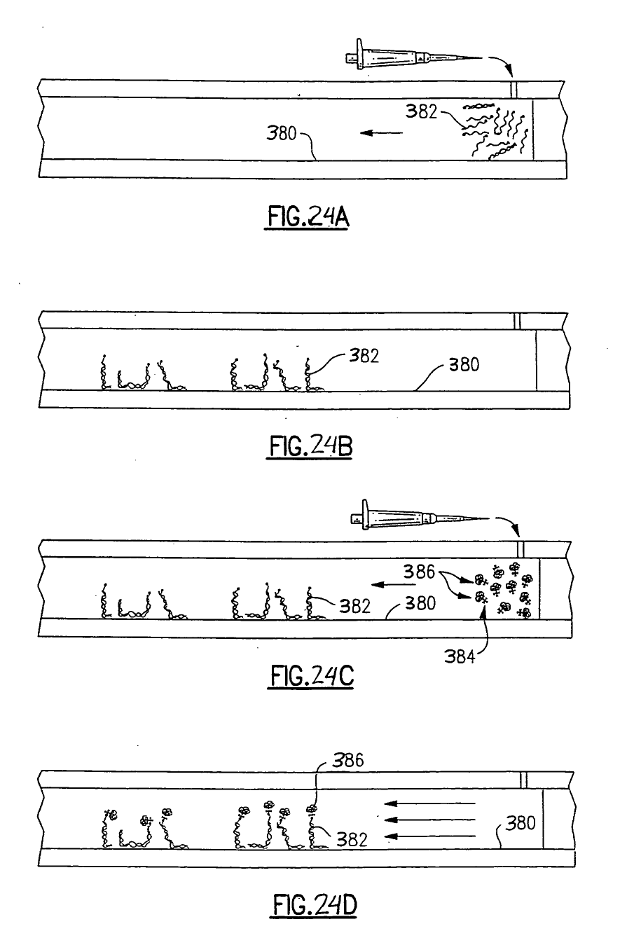

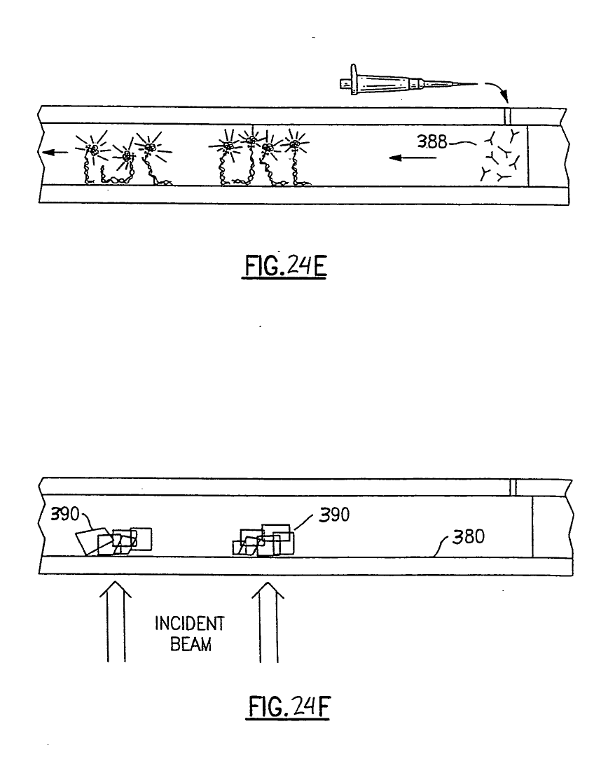

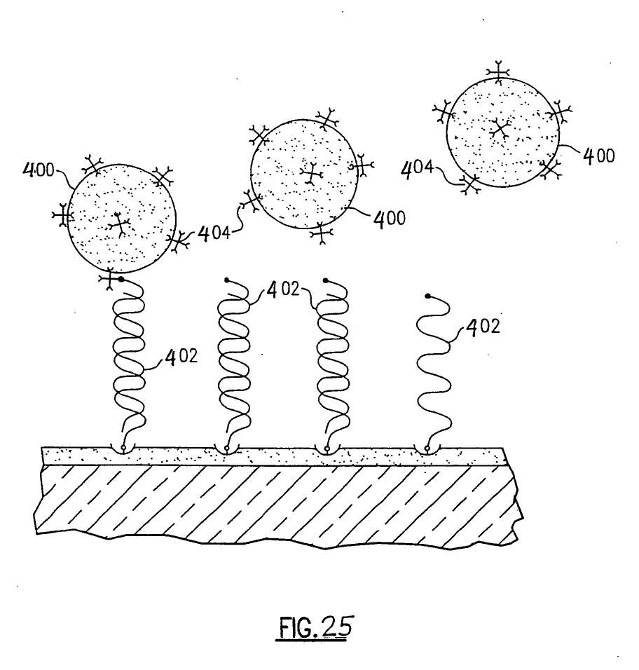

In addition to luminescence, techniques such as color staining using an enzyme-linked immunosorbent assay (ELISA) and gold labeling can be used to alter the optical properties of biological antigen material. For example, in order to test for the presence of an antibody in a blood sample, possibly indicating a viral infection, an ELISA can be carried out which produces a visible colored deposit if the antibody is present. Referring to FIGS. 24A, 24B, 24C, 24D, 24E, and 24F, an ELISA makes use of a surface 380 that is coated with an antigen 382 specific to the antibody 384 to be tested for. Upon exposure of the surface to the blood sample 386, antibodies in the sample bind to the antigens. Subsequent staining of the surface with specific enzyme-conjugated antibodies 388 and reaction of the enzyme with a substrate produces a precipitate 390 that correlates with the level of antigen binding and hence allows the presence of antibodies in the sample to be identified by the optical disc drive. This precipitate is then detected by the incident beam.

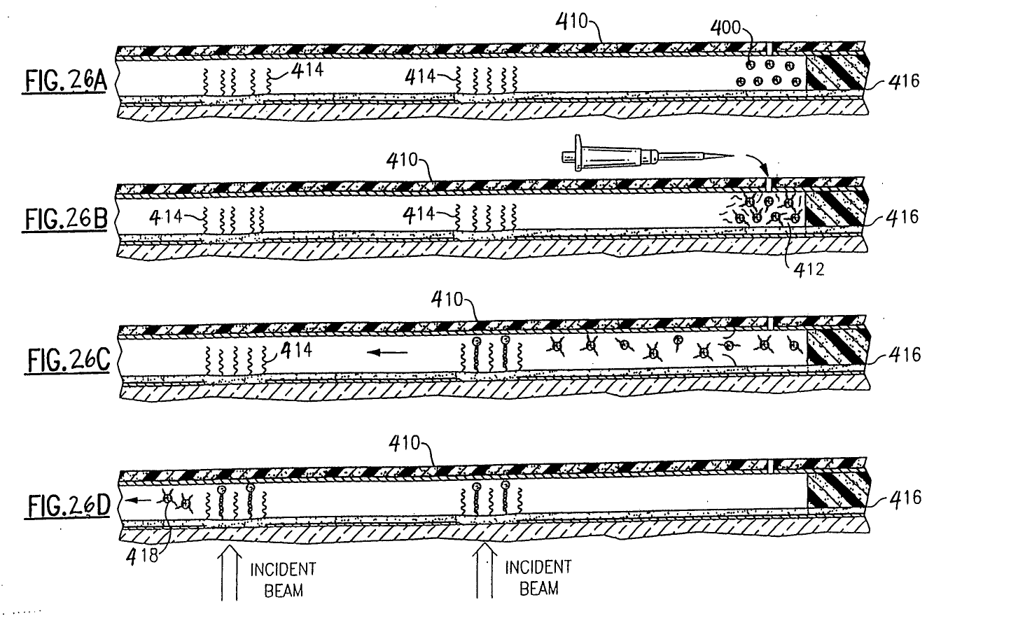

Referring to FIG. 25, bead-based assays involve use of spherical micro-particles, or beads 400 to alter the optical properties of biological antigen material 402. The beads 400 are coated with a chemical layer 404 having a specific affinity for the investigational feature in a test sample. Referring to FIGS. 26A, 26B, 26C, and 26D, when a test sample is loaded into or onto an optical disc 410 containing reporter beads 400 (FIG. 25), the investigational feature 412, if present, binds to the reporter beads 400. Investigational feature 412 further binds to specific capture agents 414 on the surface 416 of the optical disc 410. In this way, if the investigational feature is present in the biological solution, it becomes a binding agent to bind bead reporters 400 to capture agents 414 on the surface 416 of the bio-disc 410. When the bio-disc is spun in the optical disc drive, the resulting centrifugal force sends unbound bead reporters 418 to an outer periphery of the disc, while bound bead reporters remain distributed over the area of the disc coated with the capture agent. The bound beads can be detected and quantified using an optical disc reader. Related dual bead assays are further disclosed in U.S. patent application Ser. No. 09/997,741 entitled “Dual Bead Assays Including Optical Biodiscs and Methods Relating Thereto” filed Nov. 27, 2001, which is incorporated herein by reference.

Reporters useful in the invention include, but are not limited to, synthetic or biologically produced nucleic acid sequences, synthetic or biologically produced ligand-binding amino acids sequences, products of enzymatic reactions, and plastic micro-spheres or beads made of, for example, latex, polystyrene or colloidal gold particles with coatings of bio-molecules that have an affinity for a given material such as a biotin molecule in a strand of DNA. Appropriate coatings include those made from streptavidin or neutravidin, for example. These beads are selected in size so that the read or interrogation beam of the optical disc drive can “see” or detect a change of surface reflectivity caused by the particles.

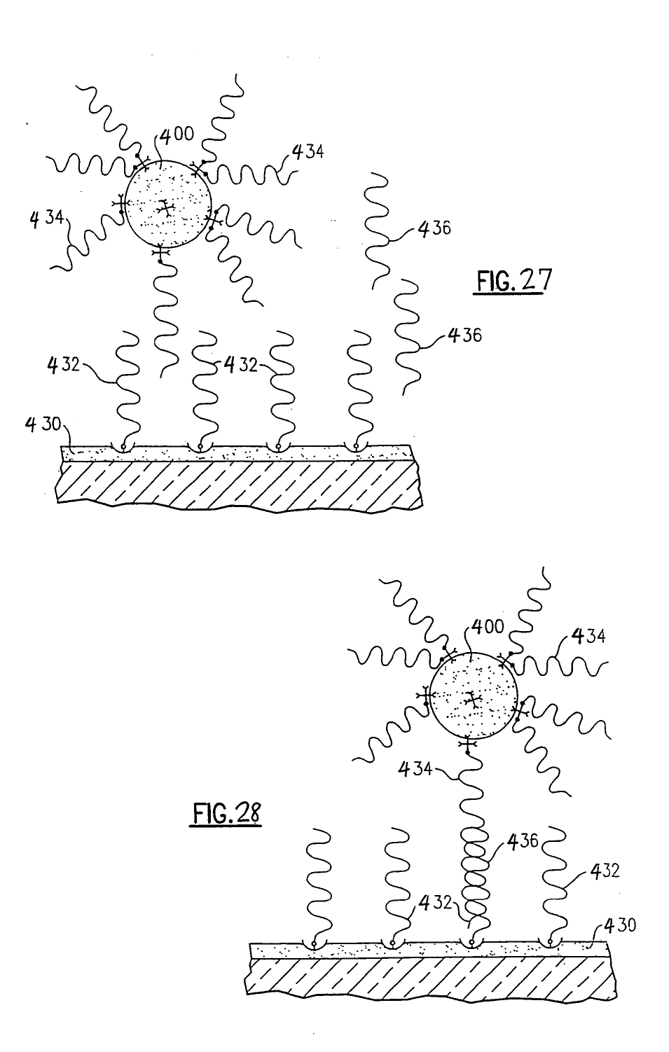

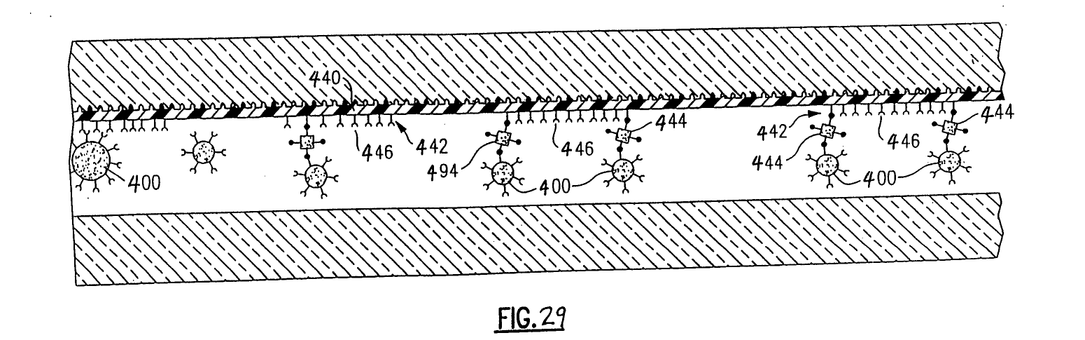

In some embodiments associated with the present invention, reporter beads are bound to the disc surface through DNA hybridization. Referring to FIGS. 27 and 28, a capture probe 432 is attached to the disc surface 430, while a signal probe 434 is attached to reporter beads 400 (FIG. 25). In the case of a hybridization assay, both of the probes are complementary to the target sequence 436. In the presence of target sequence 436, both capture and signal probes hybridize with the target. In this manner, beads 400 are attached to disc surface 430. In a subsequent centrifugation (or wash) step, all unbound beads are removed. Alternatively, the target itself is directly bound or linked to the beads without the presence of an extra signaling probe.

Referring to FIG. 29, in the case of an immunoassay, the disc surface 440 is coated with a receptor 442 (e.g., antibody), which specifically binds to the analyte of interest 444 (e.g., investigational feature). The capture zones 446 for each specific analyte to be assayed could be separated in the analysis field of the disc. If an analyte 444 (antigen or antibody) is captured by the receptor 442 (antibody or antigen, respectively), present on the capture zone 446, then a signal generation combination specific for the analyte can be used to quantify the presence of the analyte.

Alternatively, an investigational feature, if of adequate size for detection by the incident beam of an optical disc drive, may not require a reporter. Certain chemical reactions and the products and by-products resulting therefrom (i.e., precipitates), induce a sufficient change in the optical properties of the biological sample being tested. Such a change may also occur upon detection of the investigation feature itself, such as is the case when the invention is used to create an image of a microscopic structure. The optical disc drive detects changes in the optical properties of the surface of the bio-disc and creates images based thereon.

In a particular embodiment of the invention, an optical disc system (e.g., FIG. 10) includes a signal processing system and a photo detector circuit (e.g., 158 of FIG. 12) of an optical disc drive configured to generate at least one information-carrying signal (e.g., the HF, TE, or FE signals) from an optical disc assembly (e.g., disc 110 of FIG. 10). The signal processing system is coupled to the photo detector 158 to obtain from the at least one information-carrying signal both operational used to operate the optical disc system and indicia data (e.g., traces in FIG. 19B) indicative of a presence of an investigational feature associated with the optical disc assembly.

In a variant of the invention, the signal processing system of the optical disc system includes a PC and an analog-to-digital converter to provide a digitized signal to the PC. The analog-to-digital converter is coupled between the at least one information carrying signal and the PC. The PC includes a program module to detect and characterize peaks (e.g., see traces in FIG. 19B) in the digitized signal. Preferably, the PC further includes another program module to detect and count double peaks (e.g., see traces in FIG. 19B) in the digitized signal.

In another variant of the invention, the signal processing system of the optical disc system includes a PC, an analog-to-digital converter to provide a digitized signal to the PC, and an analyzer coupled between an analog-to-digital converter and a PC. The analog-to-digital converter is coupled between the at least one information carrying signal and the PC. The analyzer includes logic to detect and characterize peaks in the digitized signal. Preferably, the analyzer further includes logic to detect and count double peaks in the digitized signal.

In still another variant of the invention, the signal processing system of the optical disc system includes a PC and an analog-to-digital converter to provide a digitized signal to the PC. The analog-to-digital converter is coupled between the at least one information carrying signal and the PC. The signal processing system further includes an audio processing module coupled between the at least one information-carrying signal and the analog-to-digital converter. Preferably, the optical disc assembly is pre-recorded with a predetermined sound, and the PC includes a program module to detect the indicia data in a deviation of the at least one information carrying signal from the predetermined sound when the investigational feature is present. In an alternative variant, the predetermined sound is encoded silence.

In still yet another variant of the invention, the signal processing system of the optical disc system includes a PC and an analog-to-digital converter to provide a digitized signal to the PC. The analog-to-digital converter is coupled between the at least one information carrying signal and the PC. The signal processing system further includes an external buffer amplifier coupled between the at least one information-carrying signal and the analog-to-digital converter.

In a further variant of the invention, the signal processing system of the optical disc system includes a PC and an analog-to-digital converter to provide a digitized signal to the PC. The analog-to-digital converter is coupled between the at least one information carrying signal and the PC. The signal processing system further includes a trigger detection circuit coupled to the analog-to-digital converter. The trigger detection circuit is operative to detect a particular time in relation to a time when the indicia data is present in the at least one information-carrying signal.

In an alternative embodiment, the signal processing system includes a programmable digital signal processor selectively configurable to either (1) extract the operational information from the at least one information-carrying signal while in a first configuration or (2) operate as an analog-to-digital converter to provide the indicia data while in a second configuration.

In another alternative embodiment, the signal processing system of the optical disc system includes a PC, a programmable digital signal processor coupled to the at least one information-carrying signal, and an analyzer coupled between the programmable digital signal processor and the PC.

In yet another alternative embodiment, the signal processing system of the optical disc system includes a trigger detection circuit that detects a time period during which the investigational feature associated with the optical disc assembly is scanned by the photo detector circuit.

In a further alternative embodiment, the signal processing system of the optical disc system includes a trigger detection circuit that detects a particular time in relation to a time when the indicia data is present in the at least one information-carrying signal. The time when the indicia data is present in the at least one information-carrying signal occurs periodically. The particular time is either (1) a predetermined time in advance of, (2) a time at, or (3) a predetermined time after each time the indicia data either begins to be present or ends in the at least one information-carrying signal.

In still yet another alternative embodiment, the signal processing system of the optical disc system includes a PC, and an audio processing module coupled between the PC and the at least one information-carrying signal. Preferably, the sound processing module is either an external module independent of the optical disc drive, a drive module that is a part of the optical disc drive, or a modified drive module that is a part of the optical disc drive. In a variant of this embodiment, the PC includes a processor coupled to the sound module, and a software module stored in a memory to control the processor to extract the indicia data from sound data.

In yet a further alternative embodiment, the photo detector circuit of the optical disc system includes circuitry to generate an analog signal as the at least one information-carrying signal. The analog signal includes either a high frequency signal from a photo detector, a tracking error signal, a focus error signal, an automatic gain control setting, a push-pull tracking signal, a CD tracking signal, a CD-R tracking signal, a focus signal, a differential phase detector signal, a laser power monitor signal or a sound signal.

In another embodiment, the optical disc system further includes the optical disc assembly (e.g., 110 of FIG. 10). The optical disc assembly has the associated investigational feature disposed on the assembly in a first disc sector and has the operational information used to operate the optical disc drive encoded on the assembly in a remaining disc sector.

In a variant, the optical disc assembly includes a trigger mark (e.g., 126 of FIG. 10) that is disposed on the optical disc assembly in a predetermined position relative to the first disc sector. The signal processing system further includes a trigger detection circuit (e.g., 158 of FIG. 10) that detects the trigger mark. Preferably, the trigger detection circuit detects the trigger mark periodically and detects the trigger mark either (1) a predetermined time in advance of, (2) a time at, or (3) a predetermined time after a time when the associated investigational feature is read by the photo detector circuit based on the predetermined position of the trigger mark relative to the first disc sector.

In a variant, the associated investigational feature of the optical disc assembly includes either plastic micro-spheres with a bio-molecule coating, colloidal gold beads with a bio-molecule coating, silica beads, glass beads, magnetic beads, or fluorescent beads.

In another embodiment of the invention, there is provided a method that includes the steps of depositing a test sample, spinning the optical disc, directing an incident beam, detecting a return beam, processing the detected return beam, and processing the detected return beam. The step of depositing a test sample includes depositing the sample at a predetermined location on an optical disc assembly. The step of spinning the optical disc includes spinning the assembly in an optical disc drive. The step of directing an incident beam includes directing the beam onto the optical disc assembly. The step of detecting a return beam includes detecting the return beam formed as a result of the incident beam interacting with the test sample. The step of processing the detected return beam processes the detected return beam to acquire information about an investigational feature associated with the test sample.

In a variant of this embodiment, the step of detecting a return beam forms a plurality of analog signals. The step of processing the detected return beam includes summing a first subset of the plurality of analog signals to produce a sum signal, combining either the first subset or a second subset of the plurality of analog signals to produce a tracking error signal, obtaining information used to operate an optical disc drive from the tracking error signal, and converting the sum signal to a digitized signal.

In another embodiment of the invention, the invention is a method that includes steps of acquiring a plurality of analog signals, summing a first subset, combining a second subset, obtaining information, and converting the sum signal to a digitized signal. The step of acquiring a plurality of analog signals acquires analog signals from an optical disc assembly using a plurality of photo detectors. The step of summing a first subset sums a first subset of the plurality of analog signals to produce a sum signal. The step of combining a second subset combines a second subset of the plurality of analog signals to produce a tracking error signal. The step of obtaining information obtains information used to operate an optical disc drive from the tracking error signal.

In a variant, the steps of acquiring and summing produce the sum signal that includes perturbations indicative of an investigational feature located at a location of the optical disc assembly.

In another variant, the method further includes a step of characterizing the investigational feature based on the digitized signal.

In another variant of the method, the step of converting includes configuring a portion of an optical disc drive chip set to operate as an analog-to-digital converter. Preferably, the step of configuring includes programming a digital signal processing chip within the optical disc drive chip set to operate as an analog-to-digital converter. Preferably, the digital signal processing chip includes a normalization function, an analog-to-digital converter function, a demodulation/decode function, and an output interface function. Preferably, the step of configuring further includes passing the sum signal around the demodulation/decode function by creating a path from the analog-to-digital converter function to the output interface function. Preferably, the step of configuring further includes deactivating the demodulation/decode function.

In another variant of the method, the step of converting includes configuring a digital signal processing chip that includes a normalization function, an analog-to-digital converter function, a demodulation/decode function, and an output interface function, and the step of configuring includes creating a path from the analog-to-digital converter function to the output interface function so that the sum signal is unprocessed by the demodulation/decode function. Preferably, the step of configuring includes deactivating the demodulation/decode function.

In yet another embodiment of the invention, a method includes steps of adapting a portion of a signal processing system, acquiring a plurality on analog signals, converting the analog signals, and characterizing investigational features based on a digitized signal. The step of adapting a portion of a signal processing system includes adapting the portion to operate as an analog-to-digital converter. The step of acquiring a plurality on analog signals acquires the analog signals from a photo detector circuit of an optical disc drive. The plurality of analog signals includes information that is indicative of investigational features on an optical disc assembly. The step of converting the analog signals converts the analog signals into a digitized signal with the signal processing system. Preferably, the step of adapting includes programming a digital signal processing chip within the signal processing system to operate as the analog-to-digital converter.

In another alternative embodiment of the invention, a method includes steps of receiving and converting. The step of receiving includes receiving each of at least one analog signal at a corresponding input of signal processing circuitry. The at least one analog signal has been provided by at least one corresponding photo detector element that detects light returned from a surface of an optical disc assembly. The step of converting includes converting each of the at least one analog signal into a corresponding digitized signal. Each digitized signal is substantially proportional to an intensity of the returned light detected by a corresponding one of the at least one photo detector element.

In a variant of this embodiment, the step of converting includes operating the signal processing circuitry to bypass any demodulation of a first digitized signal. Preferably, the step of converting further includes operating the signal processing circuitry to bypass any decoding of the first digitized signal, and operating the signal processing circuitry to bypass any checking for errors in the first digitized signal.

In another variant of this embodiment, the step of converting includes operating the signal processing circuitry to bypass any decoding of a first digitized signal.

In yet another variant of this embodiment, the step of converting includes operating the signal processing circuitry to bypass any checking for errors in a first digitized signal.

In still another variant of this embodiment, the method further includes a step of combining at least two of the at least one analog signal. Preferably, the step of combining is a step selected from a group consisting of adding, subtracting, dividing, multiplying, and a combination thereof. Preferably, the step of combining is performed before the step of converting. Alternatively, the step of combining may be performed after the step of converting.

In a further variant, the method further includes a step of supplying a first digitized signal of the at least one digitized signal at an output interface of the signal processing circuitry after the step of converting without substantially modifying the first digitized signal between the steps of converting and supplying. Preferably, the signal processing circuitry includes a digital signal processor. Preferably, the signal processing circuitry consists of a digital signal processor.

The materials for use in the method of the invention are ideally suited for the preparation of a kit. Such a kit may include a carrier member being compartmentalized to receive in close confinement an optical bio-disc and one or more containers such as vials, tubes, and the like, each of the containers including a separate element to be used in the method. For example, one of the containers may include a reporter and/or protein-specific binding reagent, such as an antibody. Another container may include isolated nucleic acids, antibodies, proteins, and/or reagents described herein, known in the art or developed in the future. The constituents may be present in liquid or lyophilized form, as desired. The antibodies used in the assay kits of the present invention may be monoclonal or polyclonal antibodies. For convenience, one may also provide the reporter affixed to the substrate of the bio-disc. Additionally, the reporters may further be combined with an indicator, (e.g., a radioactive label or an enzyme) useful in assays developed in the future. A typical kit also includes a set of instructions for any or all of the methods described herein.

In a variant of this embodiment, the carrier may be further compartmentalized to include a setup optical disc containing software for configuring a computer for use with the bio-disc. Optionally, the kit may be packaged with a modified optical disc drive. For example, the kit may be sold for educational purposes as an alternative to the common microscope.

Bio-Discs with Equi-Radial Analysis Zones

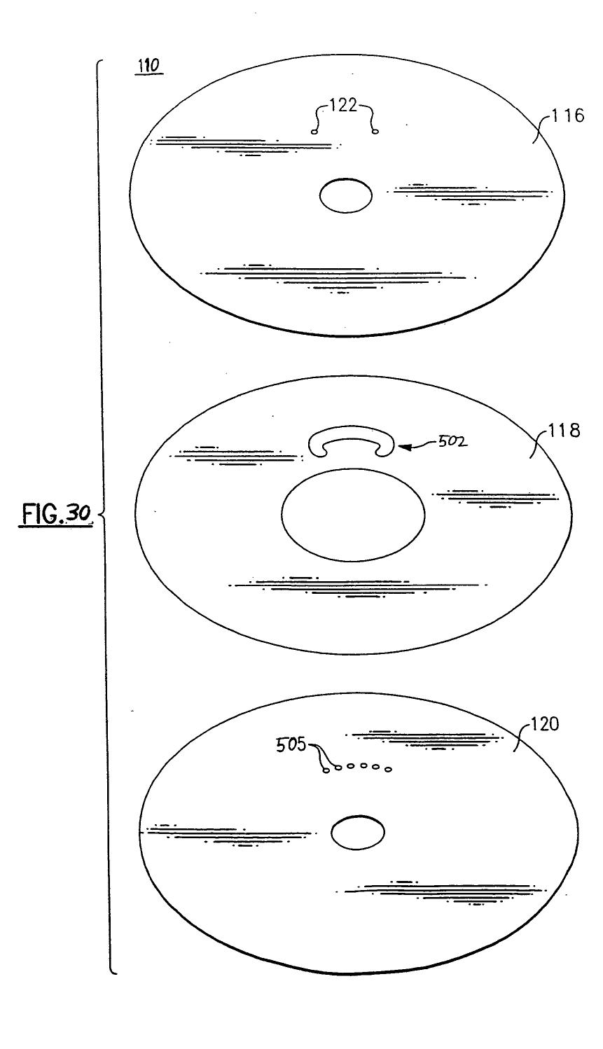

Alternative embodiments of the bio-disc according to the present invention will now be described with reference to FIGS. 30 to 35. Various features of the discs of these latter embodiments have been already illustrated with reference to FIGS. 1 to 21, and therefore such common features will not be described again in the following. Accordingly, and for the sake of simplicity, as a general rule in FIGS. 30 to 35 only the features differentiating the bio-disc 110 from those of FIGS. 1 to 21 are represented.

Furthermore, the following description of the bio-disc 110 of the invention can be readily applied to the transmissive-type as well as to the reflective-type optical bio-disc described above in conjunction with FIGS. 2-9.

Referring to FIG. 30 there is shown an exploded perspective view of the principal structural elements of one embodiment of the optical bio-disc according to the present invention, which in the present case is globally indicated by 110.

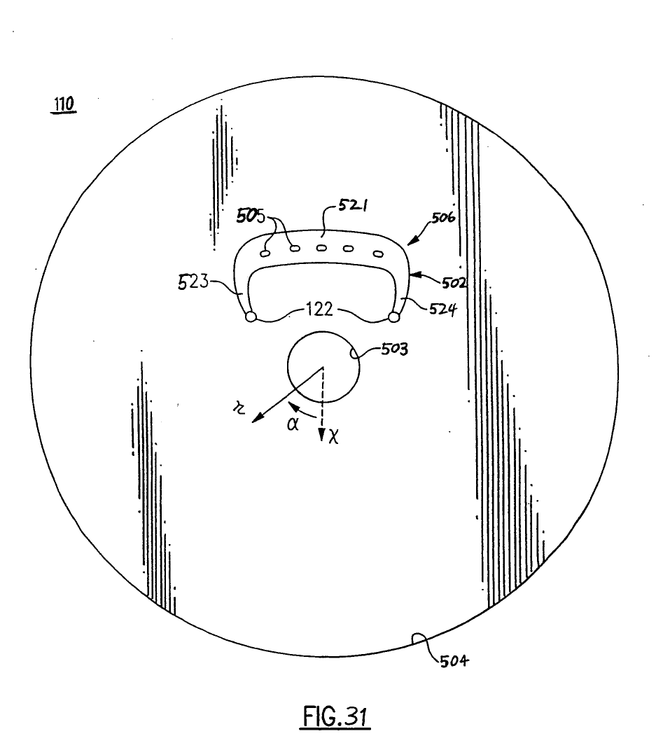

The next figure, FIG. 31 is a top plan view of bio-disc 110, wherein a cap portion 116 thereof is represented as transparent in order to reveal internal components of disc 110 itself.

With reference to FIGS. 30 and 31, optical bio-disc 110 includes the principal structural elements already introduced with reference to the preceding figures, namely the aforementioned cap portion 116, an adhesive member or channel layer 118 and a substrate 120.

The cap portion 116 includes one or more inlet ports 122. Purely by way of example and for the sake of simplicity, in FIGS. 30 and 31 only two inlet ports 122 are shown.

The adhesive member or channel layer 118 has fluid chambers 502 formed therein, in which inspection of investigational features can be conducted and which will be described in greater detail hereinbelow. Always by way of example and for the sake of simplicity, in FIGS. 30 and 31 only one fluid chamber 502 is shown.

The substrate 120 defines a circular inner perimeter 503 and a circular outer perimeter 504, concentric with the inner perimeter 503, of bio-disc 110.

The substrate 120 includes one or more reaction sites 505. In FIGS. 30 and 31 a disc including only a single set, or array, of reaction sites 505 is shown purely by way of example and for illustrative purposes only.

One of skill in the art will understand that reaction sites 505 may be in general target or capture zones. As already illustrated with reference to FIGS. 1 to 16, such target zones may be formed by physically removing an area or portion of a reflective or semi-reflective layer of the disc at a desired location or, alternatively, by masking the desired area prior to applying the reflective or semi-reflective layer. Alternatively, as already illustrated above, in the transmissive-type disc target zones may be created by silk screening ink onto the thin semi-reflective layer or they may be defined by address information encoded on the disc 110.

Bio-disc 110 also provides, at substrate 120, a series of information tracks analogous to the tracks 170 already described with reference to the embodiments of FIGS. 1 to 21 and which are therefore not represented in FIGS. 30 and 31.

In general, information tracks are of a substantially circular profile and increase in circumference as a function of radius extending from the inner perimeter 503 to the outer perimeter 504 of disc 110, typically according to a spiral profile.

Furthermore, bio-disc 110 may provide an operational layer associated with substrate 120, which layer includes encoded information located substantially along one or more information tracks, e.g. a layer analogous to the reflective layer 142 introduced with reference to FIGS. 1 to 16.

A more detailed description of fluid chamber 502 will now be provided, with reference to FIGS. 30 and 31.

First of all, it will be understood that bio-disc 110 provides, in correspondence of fluid chamber 502, an analysis area or zone, globally indicated by 506, including investigational features.

The analysis zone addressed by the present invention may include any type of reaction site(s), array(s) of spot, capture site(s) or zone(s), target zone(s), viewing window(s) and the like, and, in general, it can be any target analysis zone of whatever type, nature, and construction.

According to the general teaching of the present invention, the analysis zone 506, and therefore the fluid chamber 502, has a configuration alternative to that of the embodiments described with reference to FIGS. 1 to 16. This alternative configuration is such that when an incident beam of electromagnetic energy tracks along the information tracks, any investigational features within the analysis zone 506 are thereby interrogated following a varying angular coordinate, instead of that which is along a single radius (i.e. at a fixed angular coordinate) as in the embodiments of FIGS. 1 to 21.

As it can be easily understood and as it is shown in FIG. 31, by “angular coordinate” is herewith intended the planar angle α defined, in a plan view of disc 110, between a disc reference radial axis x and the disc radial axis r corresponding to the actual radial position of an element, e.g. an investigational feature, wherein the center of the reference system is of course set at the center of disc 110 itself. Analogously, by “radial coordinate” it is herewith intended the actual position of an element, e.g. an investigational feature, along the corresponding radial axis r.

According to a preferred embodiment, the analysis zone 506 is directed substantially along the information tracks.

In the specific embodiment shown in FIGS. 30 and 31, the fluid chamber 502 is a fluidic circuit or channel having a central portion 521 extending according to a substantially circumferential profile concentric with respect to disc inner and outer perimeter 503 and 504, and two lateral arm portions 523 and 524 extending along a substantially radial direction.

Reaction sites 505 are thus distributed along the circumferential extension of the fluid channel central portion 521, i.e. substantially along an arc of circumference. Therefore, according to the invention, reaction sites 505 are not arranged along a single radius, i.e. at a single angular coordinate, as in previous embodiments, but at a varying angular coordinate at fixed radius.

Accordingly, when an incident beam of electromagnetic energy tracks along the information tracks, the investigational features within the analysis zone 506 are thereby interrogated according to a substantially circumferential path.

In the following, this circumferential arrangement will be referred to as “equi-radial (eRad)”, and the disc providing it as an “eRad disc”. Thus, for purposes of convenience, the terms “equi-radial”, “e-radial”, “e-rad”, “eRad”, or “circumferential” may be utilized herein interchangeably.

An issue arising from the use of eRad disc 110 is the positioning of the inlet ports 122 on disc itself. As shown in FIG. 31, it is possible to have inlet ports 122 at a different radial position with respect to the circumferential portion 521 of the corresponding channel 502. However, preferably channel central portion 521 is at a higher radial coordinate with respect to the inlet ports 122, in order to prevent the centripetal forces inducing a liquid eventually contained in the channel to escape from the ports 122.

According to a variant embodiment it would also be possible to have the channel central portion at a lower radius than the inlet ports, provided that these ports are sealed, i.e. guaranteed not to leak.

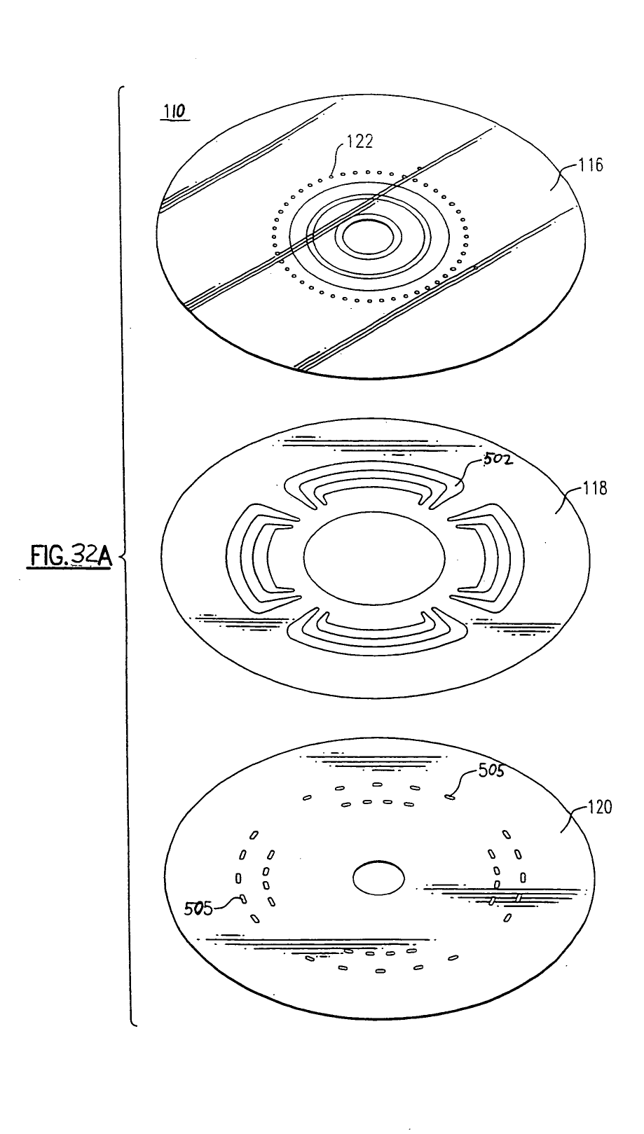

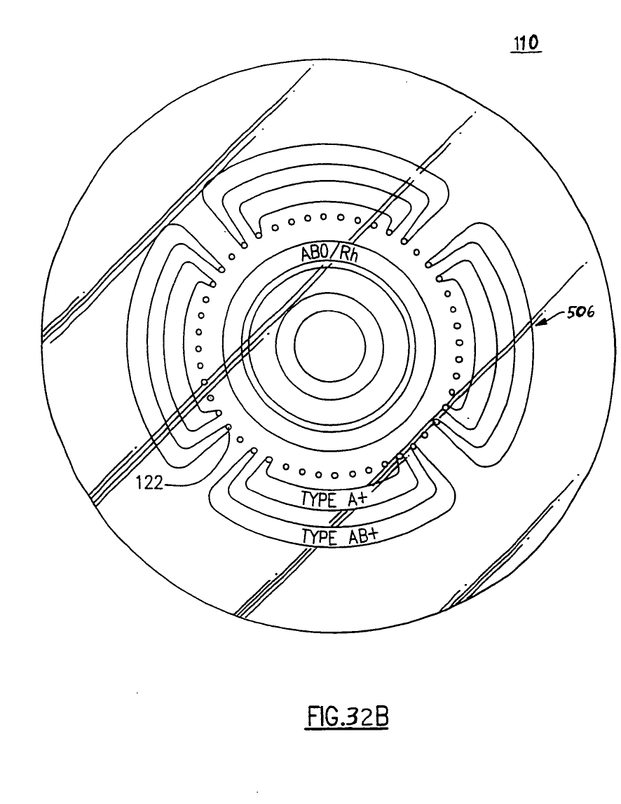

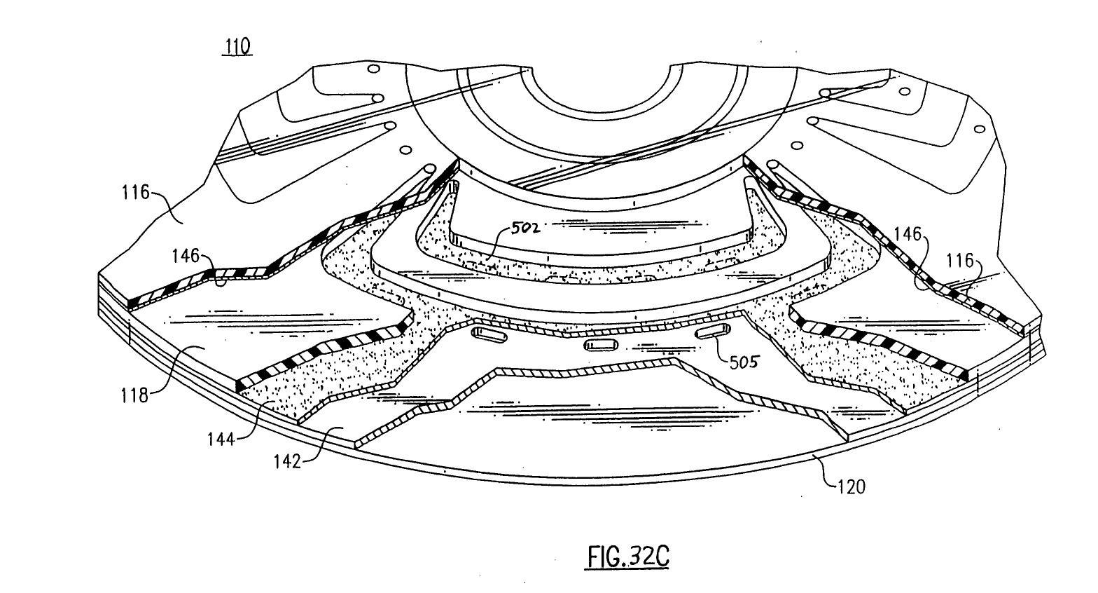

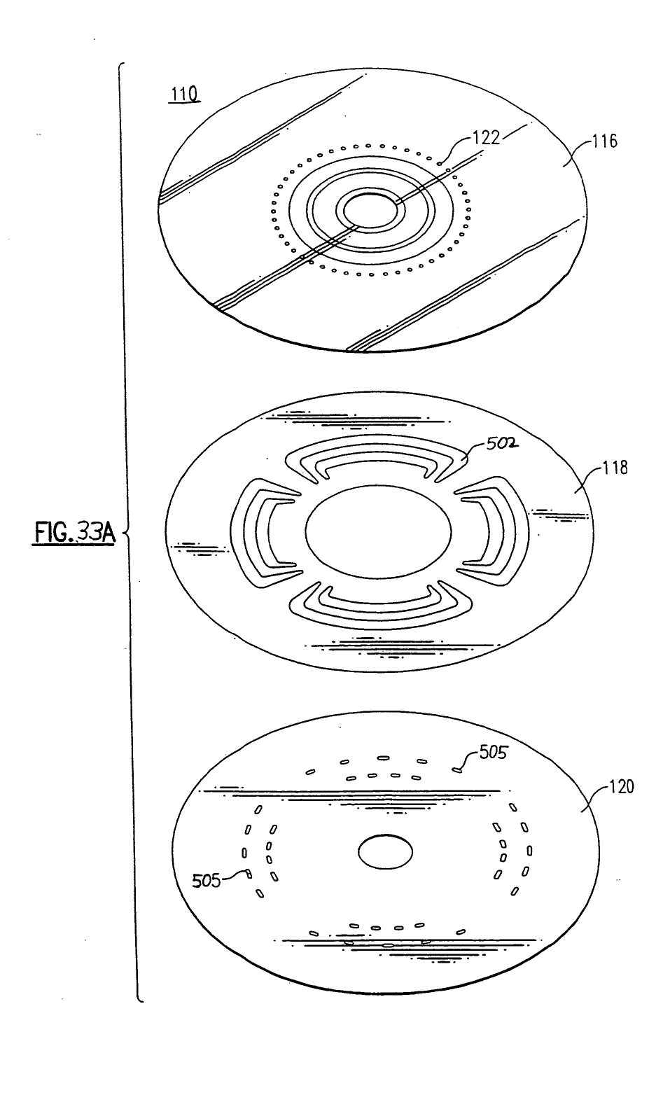

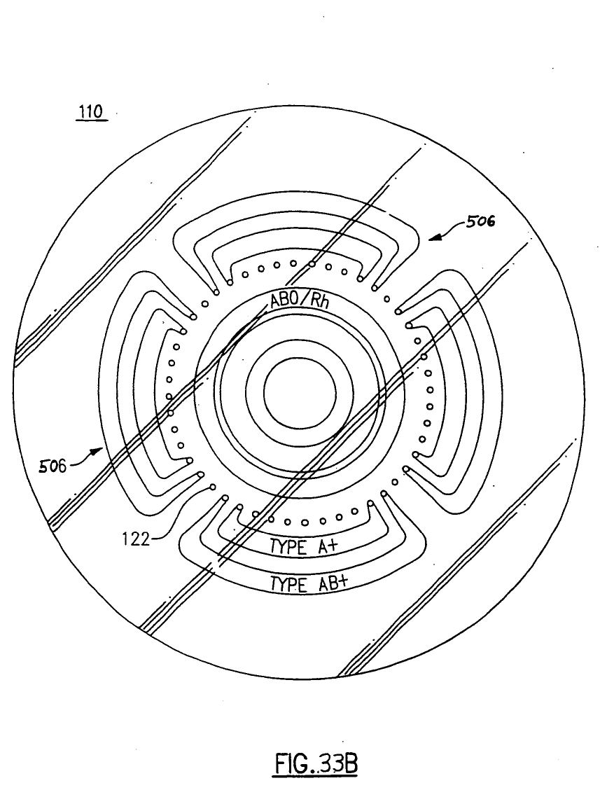

FIG. 32A is an exploded perspective view of a reflective bio-disc incorporating the equi-radial (e-rad or eRad) or circumferential channels of the present invention. This general construction corresponds to the radial-channel disc shown in FIG. 2. The e-rad implementation of the bio-disc 110 shown in FIG. 32A similarly includes the cap 116, the channel layer 118, and the substrate 120. The channel layer 118 includes the equi-radial fluid channels 502, while the substrate 120 includes the corresponding arrays of reaction sites or target zones 505.

FIG. 32B is a top plan view of the disc shown in FIG. 32A. FIG. 32B further shows a top plan view of an embodiment of the eRad disc with a transparent cap portion, which disc has two tiers of circumferential fluid channels with ABO blood type chemistry and two blood types (A+ and AB+). As shown in FIG. 32B, it is also possible to provide a priori, at the manufacturing stage of the disc of the invention, a plurality of entry ports, eventually at different radial coordinate, so that a range of equi-radial, spiraling, or radial reaction sites and/or channels are possible on one disc. These channels can be used for different test suites, or for multiple samples of single test suites.

FIG. 32C is a perspective view of the disc illustrated in FIG. 32A with cut-away sections showing the different layers of the e-radial reflective disc. This view is similar to the reflective disc 110 shown in FIG. 4. The e-rad implementation of the reflective bio-disc 110 shown in FIG. 32C similarly includes the reflective layer 142, active layer 144 as applied over the reflective layer 142, and the reflective layer 146 on the cap portion 116.

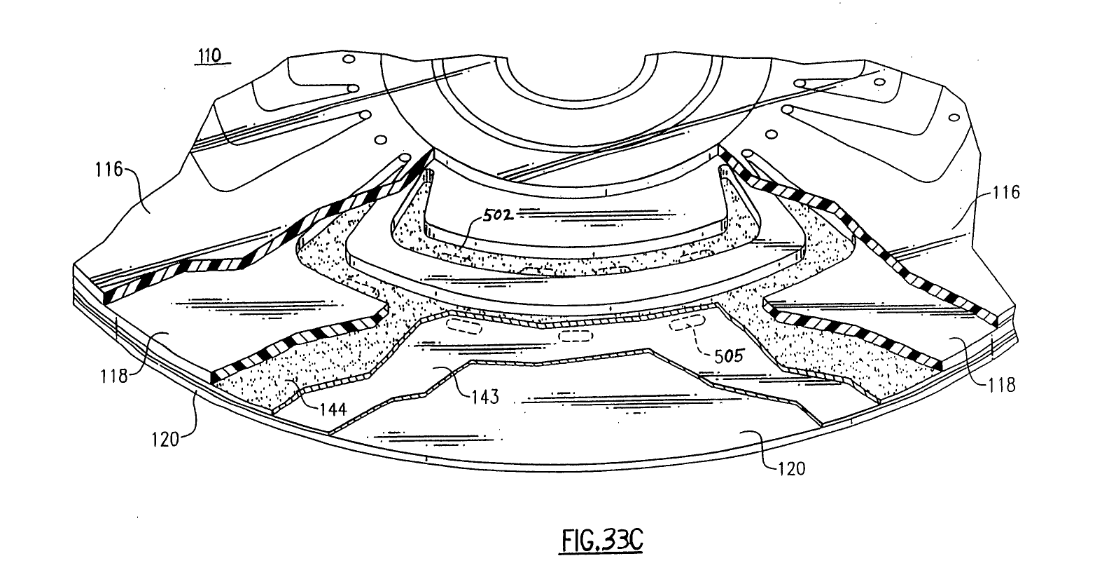

FIG. 33A is an exploded perspective view of a transmissive bio-disc utilizing the e-radial channels of the present invention. This general construction corresponds to the radial-channel disc shown in FIG. 5. The transmissive e-rad implementation of the bio-disc 110 shown in FIG. 33A similarly includes the cap 116, the channel layer 118, and the substrate 120. The channel layer 118 includes the equi-radial fluid channels 502, while the substrate 120 includes the corresponding arrays of reaction sites 505.

FIG. 33B is a top plan view of the transmissive e-rad disc shown in FIG. 33A. FIG. 33B further shows two tiers of circumferential fluid channels with ABO chemistry and two blood types (A+ and AB+). As previously discussed, the assays are performed in the analysis zones 506.

FIG. 33C is a perspective view of the disc illustrated in FIG. 33A with cut-away sections showing the different layers of this embodiment of the e-rad transmissive bio-disc. This view is similar to the transmissive disc 110 shown in FIG. 9. The e-rad implementation of the transmissive bio-disc 110 shown in FIG. 31C similarly includes the thin semi-reflective layer 143 and the active layer 144 as applied over the thin semi-reflective layer 143.

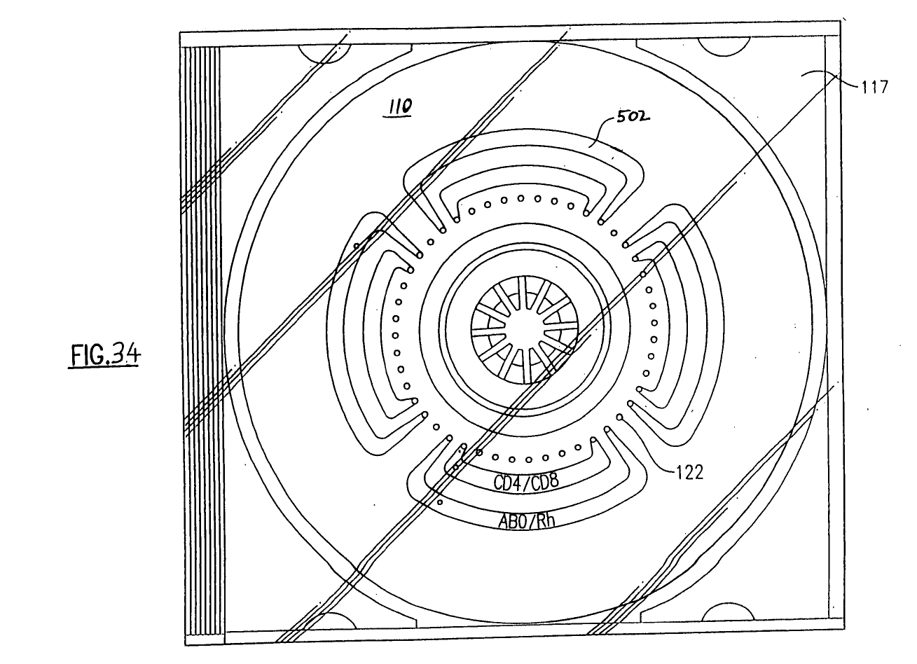

FIG. 34 shows a top plan view of an embodiment of eRad disc with a transparent cap portion, which disc has two tiers of circumferential fluid channels with two different assays, namely CD4/CD8 chemistry and ABO/RH chemistry. The disc 110 is illustrated in a bio-safe jewel case 117.

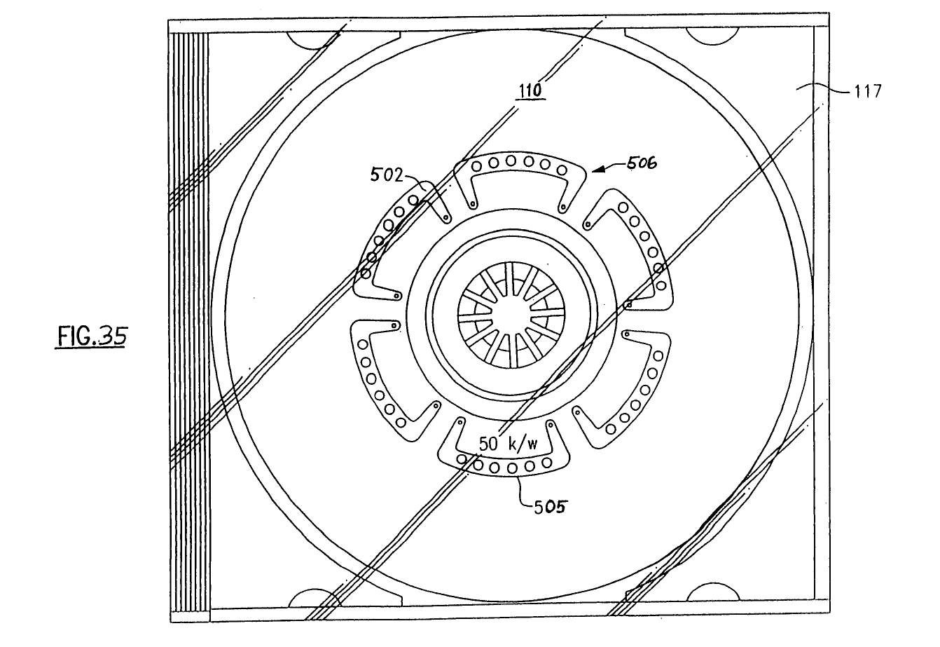

FIG. 35 shows a top plan view of an embodiment of CD4/CD8 eRad disc with a transparent cap portion, which has six circumferential fluid channels or Erad channels arranged at substantially the same radial. The disc 110 of FIG. 35 is also illustrated in the bio-safe jewel case 117.

The present invention also provides an optical analysis disc drive system of the type described in conjunction with FIGS. 1 and 10, including interrogation means of the investigational features, and in particular the light source, optical detector(s) and associated optical components already described above in conjunction with FIG. 10.

According to the invention, the interrogation means are adapted to interrogate the investigational features within the disc analysis zone according to a varying angular coordinate, and preferably circumferentially or spirally.

Preferably, the arrangement of the disc and of the system is such that rotation of the disc itself distributes investigational features in a substantially consistent distribution along the chamber.

More preferably, rotation of the disc distributes the concentration of investigational features in a substantially even distribution along the analysis chamber.

The invention also provides an analysis method using a bio-disc and an optical disc drive system as described so far, which method provides an interrogation step of the disc investigational features such that when an incident beam of electromagnetic energy tracks along disc information tracks, any investigational features within the analysis zone are thereby interrogated according to a varying angular coordinate, and in particular according to a circumferential or spiral path.

Detection of Hemoglobin and Glycohemoglobin using the Optical Bio-Disc

Glycohemoglobin analysis is used in long-term carbohydrate control of diabetics. Glycohemoglobin is formed when glucose binds to hemoglobin (Hb) at the N-terminal valine on the beta-chain resulting in the formation of HbA1c. Antibody-based assays have been used to detect the non-enzymatic glycation of Hb directly. However, producing HbA1c specific antibodies in animals is very difficult since the sugar moiety of the glycohemoglobin molecule is not exposed and will rarely result in a specific immuneresponse. A combination of isocratic ion exchange chromatography with a class-specific immunoassay for hemoglobin can rapidly analyze glycated hemoglobin without the need of a specific probe for HbA1c. Different methods for glycohemoglobin analysis implemented on the optical bio-discs are described below.

Cation Exchange Linked Immunoassay (CELIA) on the Optical Bio-Disc; Ion Exchange Resins

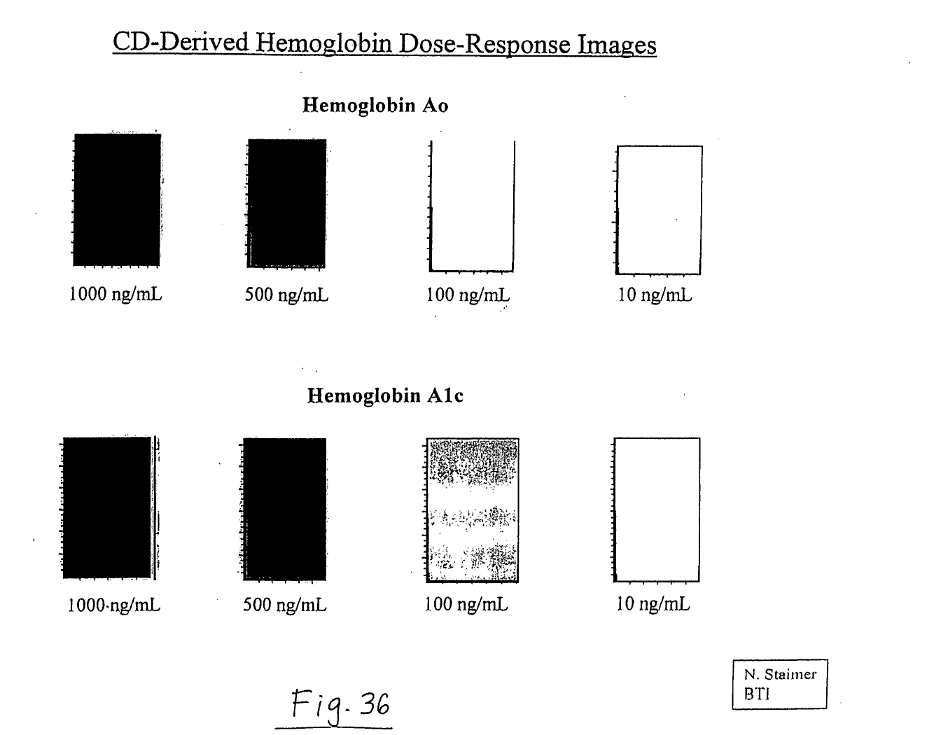

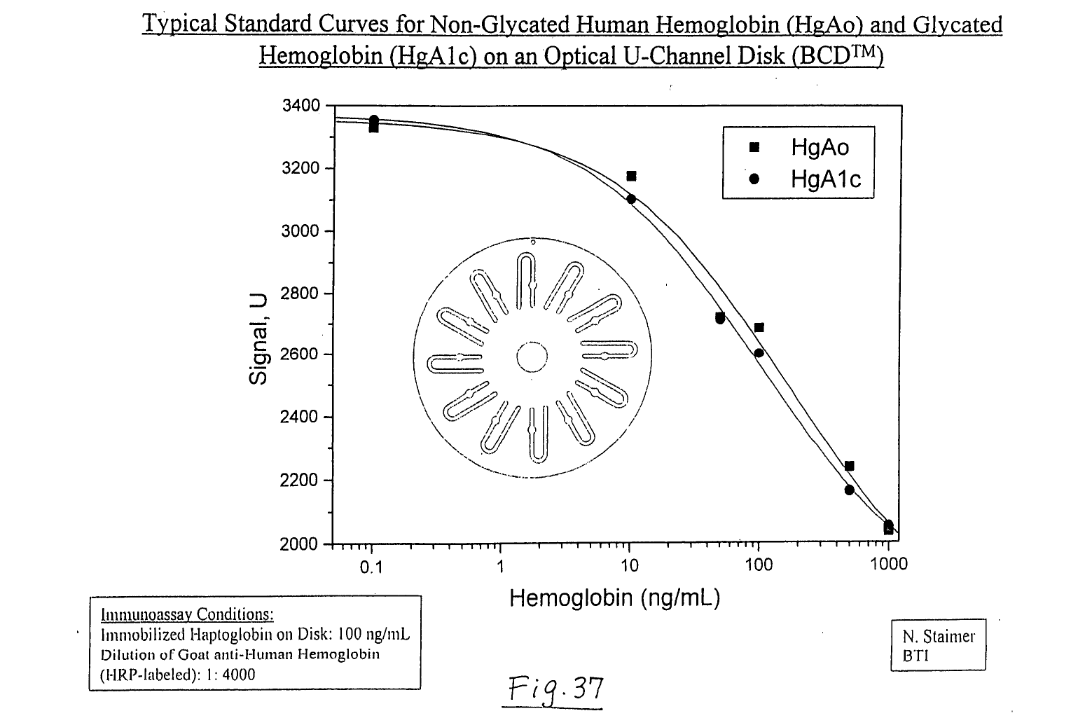

A sandwich immunoassay for hemoglobin was developed by immobilizing haptoglobin (a general capture agent for hemoglobin species) directly on the gold surface or reflective layer 143 of the optical bio-disc substrate 110. Horseradish peroxidase (HRP)-labeled goat anti-human hemoglobin antibody was used as the enzyme conjugated signal antibody. ABTS [2,2′-azino-di-(3-ethyl-benzthiazoline sulfonic acid)] was used as the enzyme substrate. Optical bio-disc images of the analysis chambers were taken and four-parameter-fitted standard curves were generated as shown in FIGS. 36 and 37. The results indicate that the optical bio-disc assay is sensitive for hemoglobin and is capable of detecting both glycated and non-glycated hemoglobin species to the same degree.

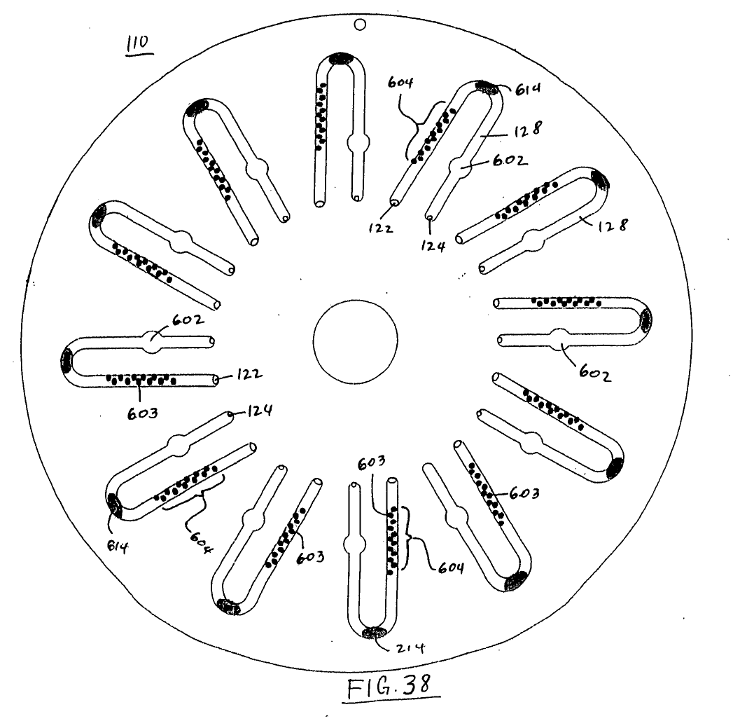

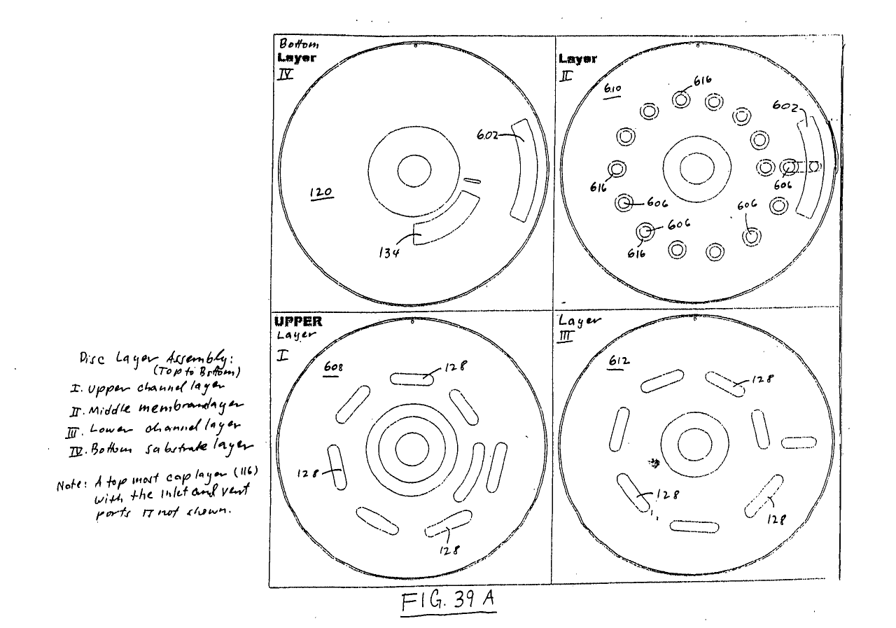

Weak cation exchange resins (e.g., carboxymethyl Sephadex beads) may be used to separate non-glycated hemoglobin from glycated hemoglobin species in a test sample. FIG. 38 illustrates an embodiment of the optical bio-disc of the present invention wherein weak cation exchange beads 603 are integrated into the fluidic circuit 128 to form a micro-chromatographic matrix 604 in the optical bio-disc 110 to isolate desired analytes including glycated hemoglobin, for example. In this method, a hemoglobin sample (e.g. blood lysate), containing both glycated and non-glycated forms of hemoglobin, is loaded into the inlet port 122. The disc 110 is then spun thereby moving the sample through the cation exchange micro-chromatographic matrix 604. The non-glycated hemoglobin binds to the beads 603 and only the glycated hemoglobin leaves the matrix 604 and moves through a filter 614 and into an analysis or assay zone 602 where the analyte is quantified as described above. Alternatively, the non-glycated hemoglobin may be isolated using anionic beads. In this alternative embodiment, glycated hemoglobin bind to the anionic beads while the non-glycated hemoglobin passes through the micro-chromatographic matrix 604 and is quantified. The total hemoglobin also needs to be quantified along with either the glycated or non-glycated hemoglobin to determine the percentage of glycated hemoglobin. The total hemoglobin may be quantified directly using the sample loaded directly into the analysis zone 602 or neutral beads may also be used in the micro-chromatographic matrix 604 wherein both forms of hemoglobin can freely pass through thereby allowing quantitation of the total hemoglobin.

Fluorescent labels may be used instead of HRP-labeled anti-human hemoglobin signal antibodies and the assay quantified using a fluorescent optical bio-disc drive. Alternatively, the capture and signal agents may be haptoglobin instead of antibodies. In this case, the assay will consist of a haptoglobin capture agent immobilized on a capture or target zone within an analysis chamber and a HRP- or fluorescent labeled haptoglobin signal agent. Other detectable labels known in the art can also be applied. The pseudo-peroxidase activity of hemoglobin can also be used to produce a detectable signal with the appropriate peroxidase substrate and requires only the (unlabeled) haptoglobin capture agent (or other capture proteins for hemoglobin) to capture the analyte, as described above.

The ion exchange matrix may be packed into the fluidic channels and separated from the analysis chamber 602 by using a different channel and/or chamber thickness for the analysis chamber 602. For example, 40-120 micron cation exchange beads may be used to form the ion exchange matrix. Thus a channel or chamber on the disc with a thickness of >120 microns (“ion exchange zone”) connected to a second channel or chamber with a thickness of <40 microns (analysis chamber) can be used. The narrower thickness of the analysis chamber prevents the beads from entering the analysis chamber. Furthermore a microfluidic channel design with a capillary valve system can also be used in conjunction with the ion exchange linked immunoassay embodiments of the present invention.

Ion Exchange Membranes

1) Lateral Flow Membranes

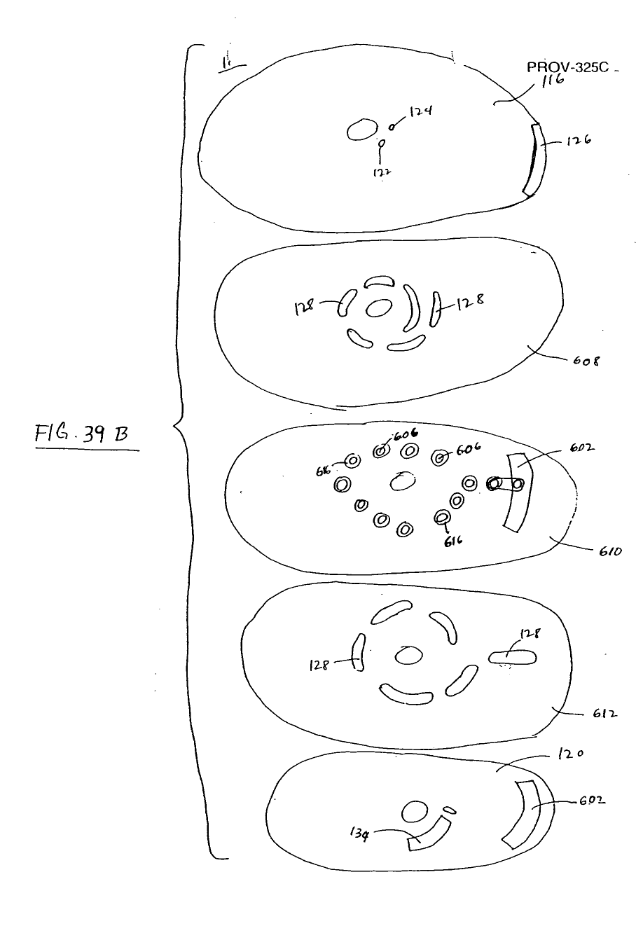

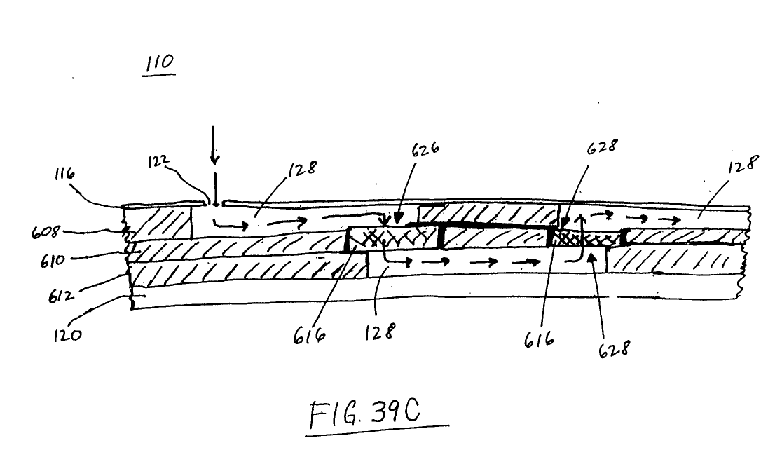

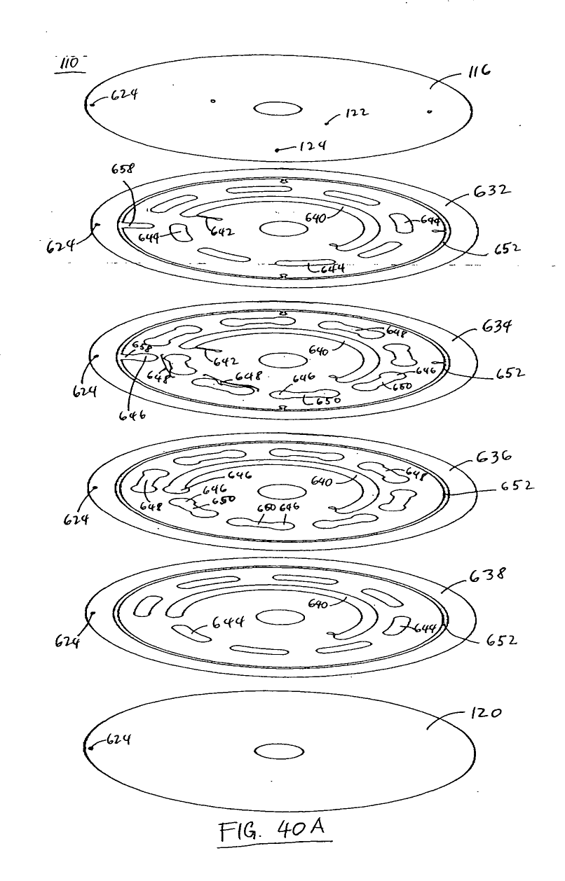

FIGS. 39 and 40 show two embodiments optical bio-discs 110 that may be used in conjuntion with the membrane chromatographic assay of the present invention wherein chemically modified membranes 616 having binders directed to either glycated or non-glycated hemoglobin, for example, may be used as the matrix material of the present invention. In this case the lateral flow membrane 616 may be formed, for example, from carboxymethyl (a weak cation) membranes, for binding non-glycated hemoglobin. The bio-disc 110 of the invention, as described below in connection with FIGS. 39 and 40, can be readily applied to the transmissive-type as well as to the reflective-type optical bio-disc described above in conjunction with FIGS. 2-9.

In a sandwich assay format method of the present invention, the capture agent, which can be an antibody or haptoglobin or another capture protein for hemoglobin, may be labeled with reporter particles (latex beads, gold beads, carbon beads, or others). After sample application and disc spinning steps, non-glycated hemoglobin binds to the cation exchange matrix and glycated hemoglobin will move to the specific analysis chamber and to the target or capture zone. The target zone is then analyzed for the presence and amount of reporter particles using the optical bio-disc reader. For the measurement of non-glycated hemoglobin the ion exchange matrix may be formed from a weak anion exchange membrane.

2) Flow Through Membrane (Membrane Adsorbers)

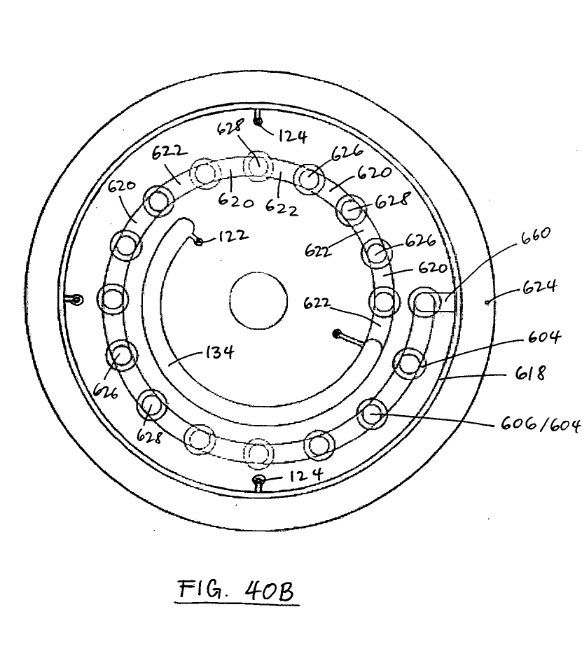

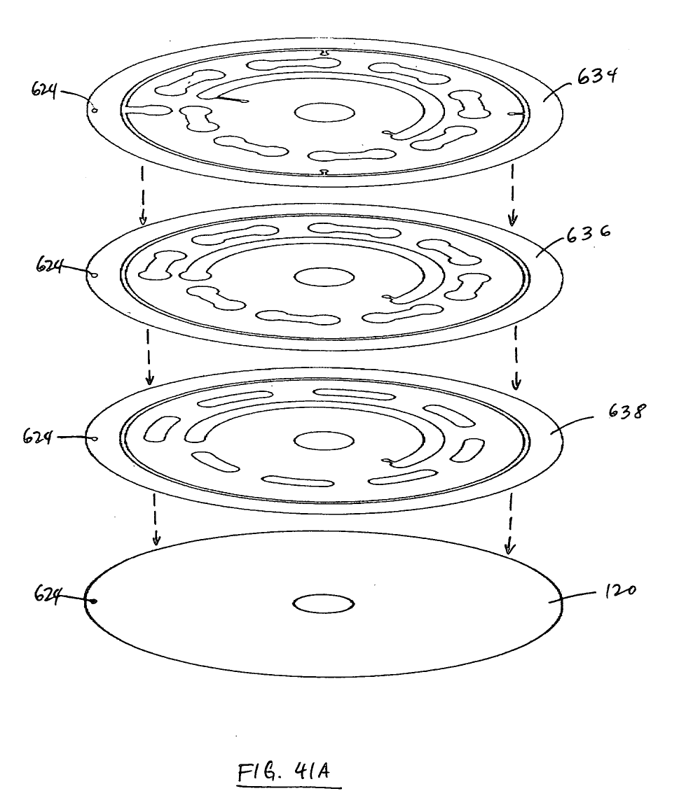

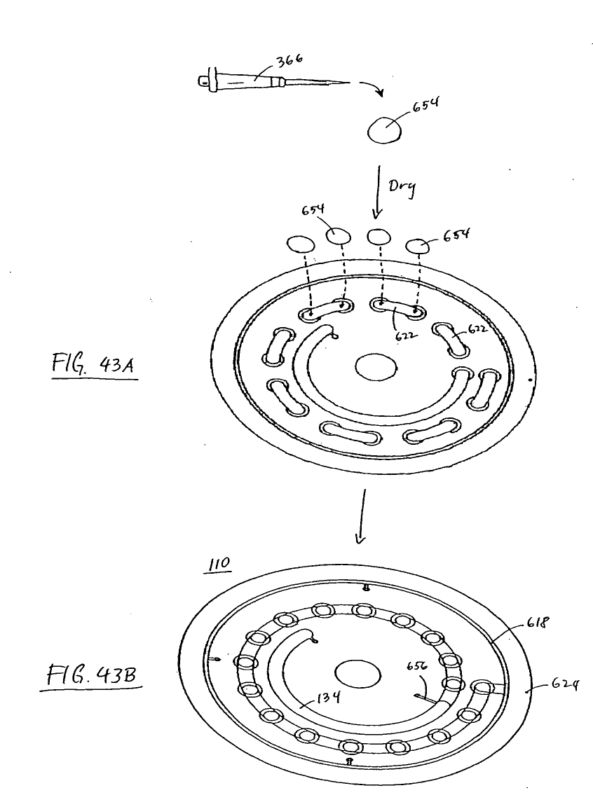

Ion Exchange Membrane Adsorbers used in ready-to-use filters (Sartorius, Goettengen, Germany) may also be used to form the matrix. Furthermore, centrifuge based Ion Exchange Membrane Spin Columns such as for example Vivapure (Vivascience, Hannover, Germany) can also be embedded into an optical bio-disc, as illustrated and described below in conjunction with FIGS. 41 and 43, and used for the separation of different isoforms of proteins (including various hemoglobin species) with subsequent, immunoassay-based optical bio-disc detection.

With reference to FIG. 39A, there is shown different layers of the bio-disc 110 for use in the lateral flow and flow through membrane based assays of the present invention. In this embodiment, several layers may be assembled to form the spiral fluidic circuit 128 as best illustrated in FIG. 40B. These layers may include a top cover disc or cap portion 116 (illustrated in FIG. 39B), an upper channel layer 608, a lower channel layer 612, a middle membrane or chromatography layer 610 situated between upper layer 608 and lower layer 612, and a bottom substrate layer 120. Substrate layer 120 may be the transmissive or reflective type substrate 120 as discussed above. The top cap portion 116 includes one or more inlet ports 122 and one or more vent ports 124 as shown in FIGS. 2, 5, 32A, and 33A. The chromatography layer 610 includes pass through ports 606 formed therein. The chemically modified membranes 616 may be placed over the pass through ports 606. The upper 608 and lower 612 channel layers have fluidic circuits 128 formed therein such that when the disc 110 is assembled with the chromatography layer 610 placed between the upper 608 and lower 612 channel layers, and the bottom substrate layer 120 and top cap portion 116 are accordingly bonded to the disc; a spiral fluidic chromatographic circuit is formed.

Referring now to FIG. 39B, there is depicted an exploded view of the bio-disc 110 described above in conjunction with FIG. 39A showing the various layers of the bio- disc including the top cap portion 116, the upper channel layer 608, the chromatography layer 610, the lower channel layer 612, and the bottom substrate layer 120.

Turning next to FIG. 39C, there is illustrated a partial cross section of a fully assembled bio-disc as described in FIGS. 39A-39C showing the direction of fluid flow (arrows) through the fluidic circuit 128. Sample is introduced into the disc 110 through the inlet port 122 of the cap portion 116. The upper channel layer 608, chromatography layer 610, and lower channel layer 612 are positioned such that fluid is directed through a series of chemically modified membranes 616 as the fluid or sample moves through the fluidic circuit 128 as illustrated. The chemically modified membranes 616 are placed over the pass through ports which include inlet passages 626 and outlet passages 628. The chemically modified membrane 616 may include for example the Ion Exchange and Lateral Flow Membranes described above.

Bioseparation with a porous membrane is of critical importance in molecular biology assays. The present application demonstrates fluidic channel arrangements for integration of porous materials, such as a porous membrane or a chromatographic membrane, into the optical bio-disc 110.

The bio-disc 110 is preferably made from several layers of polycarbonate discs and patterned adhesives to form a spiral fluidic circuit as illustrated in FIGS. 39 and 40. By integrating the porous membrane in the fluidic circuit as illustrated in FIGS. 39 and 40, the applied analyte will flow through the porous material when the analyte is driven by centrifugal and/or other types of forces.

With continuing reference to FIG. 39C, there is shown a pattern for each layer of a disc for use in biochemical assays. The optical bio-disc 110 of the present invention may include the following layers:

1) Substrate Layer 120 is a lens disc with signal tracks. The substrate layer may be a CD, CD-R, DVD, or DVD-R type disc, for example. The substrate 120 may include a reflective layer 142 which can be transmissive or partially reflective as described above in conjunction with FIGS. 2-9. Thus, it can be used to track disc spinning and provide enough optical signal for detection.

2) Lower channel layer 612 may be formed from an adhesive with fluidic channels 128 formed therein.

3) Chromatographic layer 610 is a disc layer having pass through ports 606 designed such that a chromatographic membrane material 616 may be integrated into the optical bio-disc 110. Chromatographic membranes 616 are preferably placed in or on the pass through ports 606. The membrane and chromatographic layer thickness are preferably identical. If the thickness of the membrane and chromatographic layer is different, then thickness of each can be adjusted by applying multiple layers.

4) Upper channel layer 608 may be formed from an adhesive with fluidic channels formed therein. The patterned fluidic channels overlap with the fluidic channels from the lower channel layer 612 at the pass through ports 606 of the chromatographic layer only, as shown. Thus, the analyte will pass through these fluidic paths by vertically flowing through the membranes only, as best illustrated in FIG. 39C.

5) The topmost cap portion 116 is a cover disc. The fluidic channels 128 are made to accommodate the test sample, especially when a large analyte volume is required for the assay.

6) The optical bio-disc of the present invention may optionally include a sealing layer (not shown) over the cap portion 116. It covers the vent port 124 and inlet port 122 and prevents contamination of the fluidic circuits 128 and also prevents evaporation of the test sample when loaded into the bio-disc.

Generally, the separation concept is based on having the chromatographic membrane material 616 arranged within the two layers of fluidic path as shown in FIG. 39C. Furthermore, bioseparation can be achieved by properly arranging the fluidic path to allow the analyte to flow through a series of chromatographic membranes 616.

FIG. 39C shows one segment of the integration arrangement and this design module can be scaled-up or scaled-down, by considering such factors as: membrane size and thickness, number of membranes needed, and required fluidic space.

By extending this module in series, the analyte can flow through more than two layers of membrane (as shown in FIGS. 39A-39C). The present invention may be used for example, in hemoglobin separation using a cation exchange membrane as discussed above. The present invention may also be used in various bioseparation and analyte capture applications which are different from separation by porous sizing only.

With reference now to FIG. 40A, there is shown different layers of an alternate embodiment of the bio-disc 110 for use in various assays of the present invention. In this embodiment, six layers may be assembled to form a spiral fluidic circuit having upper flow chambers 620 and lower pass through chambers 622 connected by inlet passages 626 and outlet passages 628, as best shown in FIG. 39C. These layers may include a top cover disc or cap portion 116, a first channel layer 632, a second channel layer 634, a third channel layer 636, a fourth channel layer 638, and a bottom substrate layer 120. Substrate layer 120 may be the transmissive or reflective type substrate 120 as discussed above. The top cap portion 116 includes one or more inlet ports 122 and one or more vent ports 124. In the embodiment of FIG. 40A, the first channel layer 632 has cut-out portions including an extended arcuate cut-out 640, short arcuate cut-outs 644, an inlet channel cut-out 642, a radially directed cut-out 658, and a circumferential cut-out 652. The second channel layer 634 has cut-out portions including an extended arcuate cut-out 640, a circumferential cut-out 652, an inlet channel cut-out 642, a radially directed cut-out 658 with a circular cut-out 646, and dumbell segments 648 including a central cut-out portion 650 and circular cut-outs 646 at each end thereof, as illustrated. Similarly, the third channel layer 636 includes similar cut-out portions as the second channel layer 634 without the inlet channel cut-out, as shown in FIG. 40A. The next layer is the fourth channel layer 638 which has similar cut-out portions as the first channel layer 632 without the radially directed cut-out portion. The cut-out portions from the second, third and fourth channel layers are in register with each other such that when the disc is assembled, as shown in FIG. 40B, a spiral fluidic circuit is formed including inlet ports, a mixing chamber 134, upper flow chambers 620, lower pass-through chambers 622, inlet passages 626, outlet passages 628, and a circumferential analysis chamber 618. Chemically modified membranes 616 may be placed over the inlet 626 and outlet passages 628 as best illustrated in FIG. 39C.

In yet another alternate embodiment, three layers may be assembled, instead of six, to form the spiral fluidic circuit having upper flow chambers 620 and lower pass through chambers 622 connected by inlet passages 626 and outlet passages 628. These layers may include a top cover disc or cap portion 116, a chamber layer, and a bottom substrate layer 120. Substrate layer 120 may be the transmissive or reflective type substrate 120 as discussed above. The top cap portion 116 includes one or more inlet ports 122 and one or more vent ports 124 as shown in FIGS. 2, 5, 32A, and 33A. In this embodiment of the optical bio-disc of the present in invention, the top cap portion 116 may include the upper flow chambers 620 formed therein. While the lower pass through chambers are formed in the chamber layer. The middle chamber layer may formed similar to layer 638 as shown in FIG. 40A. The chemically modified membranes 616 may be placed over the inlet passages 626 and outlet passages 628.

With continuing reference to FIG. 40B, there is shown a pictorial representation of a top plan view of the optical bio-disc shown in FIG. 40A. The various components of the disc are shown including inlet ports 122, mixing chamber 134, the upper flow chambers 620, the lower pass-through chamber 622, the chemically modified membranes 616 placed over the inlet 626 and outlet passages 628, the circumferential analysis chamber 618, and vent ports 124.

Referring now to FIGS. 41A, 41B, and 41C, there is illustrated a method for manufacturing the optical bio-disc for use in chromatographic assays. The first step in the manufacturing the bio-disc is the assembly of the second, third and fourth channel layers, and the substrate 120 as illustrated in FIG. 40A. The different layers are aligned together using the alignment hole 624 to place the various cut-out portions in register with each other. Chemically modified membranes 616 are then placed within the circular cut-out portion as illustrated in FIG. 41B. Once the membranes are in place, the remaining top layers (cap 116 and first channel layer 632) are assembled and placed over the pre-assembled bottom layers as shown in FIG. 41C, thereby completing the assembly of the optical bio-disc of the present invention as best depicted in FIG. 40B.

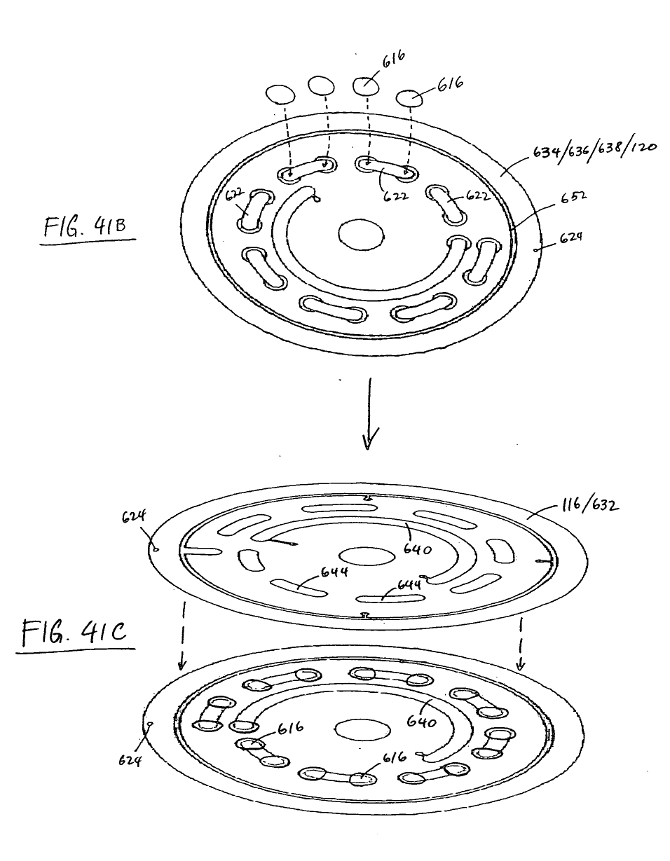

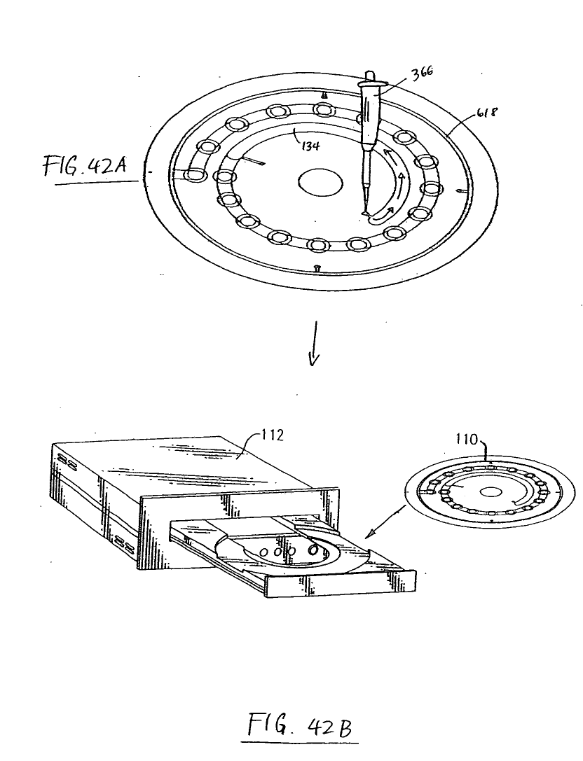

Turning next to FIGS. 42A and 42B, there is illustrated a method of using the optical bio-disc made as described in conjunction with FIGS. 41A-41C. Sample is loaded into the disc through the inlet port into the mixing chamber 134 (arrows). After the sample is loaded, the disc 110 is placed in the optical disc drive 112. The disc 110 is then spun at a pre-determined speed and time using the optical disc drive 112 and the appropriate software to control the disc rotation speed, acceleration, and time. As the disc spins, the sample moves by centrifugal force through the upper flow chambers 620 and lower pass-through chambers 622 through the inlet 626 and outlet passages 628. Since the inlet and outlet passages contain chemically modified membranes 616, specific analytes are then captured in the membranes while other analytes move through to the analysis chamber 618. The analytes in the analysis chamber are then analyzed and quantitated using a combination of signal agents, capture agents, enzymes, and or substrates to produce a signal detectable by the optical disc drive 112. The analytes bound to the membranes may also be detected and quantitated using appropriate signal antibodies or probes having attached thereto a detectable signal agent or an enzyme that can produce a detectable signal. Further details relating to assays using the optical bio-disc of the present invention are described below in Example 1.

Referring to FIGS. 43A and 43B, there are shown steps for a method of making the optical bio-disc for use in immuno-chemical or genetic assays (depending on the type of capture agent or probe used). The first step in the process is the application of a solution of capture probes onto the biological matrix or biomatrix 654. This step is followed by binding the capture probes onto the biomatrix 654 by drying the capture probe solution on the biomatrix 654. The capture agents or probes may be an antigen, antibody, ligand, receptor, binding agents, DNA, RNA, any molecule that can bind to the target or analyte, or any molecule in which the analyte specifically binds to. The individual bio-matrix pads 654 having capture probes bound thereto are then placed on a partially assembled bio-disc as illustrated. The components and assembly of this partially assembled bio-disc are described above in conjunction with FIG. 41A. After the bio-matrix pads are in place, the first channel layer 632 and top cover disc 116 are then applied as shown in FIG. 41B to complete the assembly of the bio-disc for immuno-chemical or genetic assays.

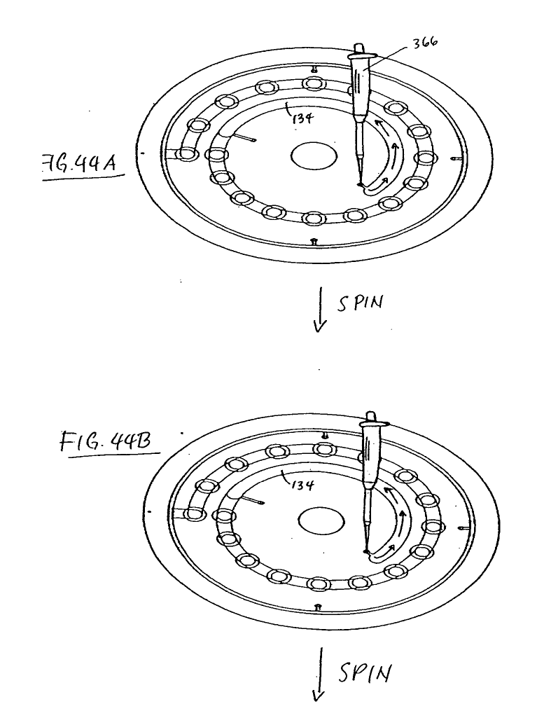

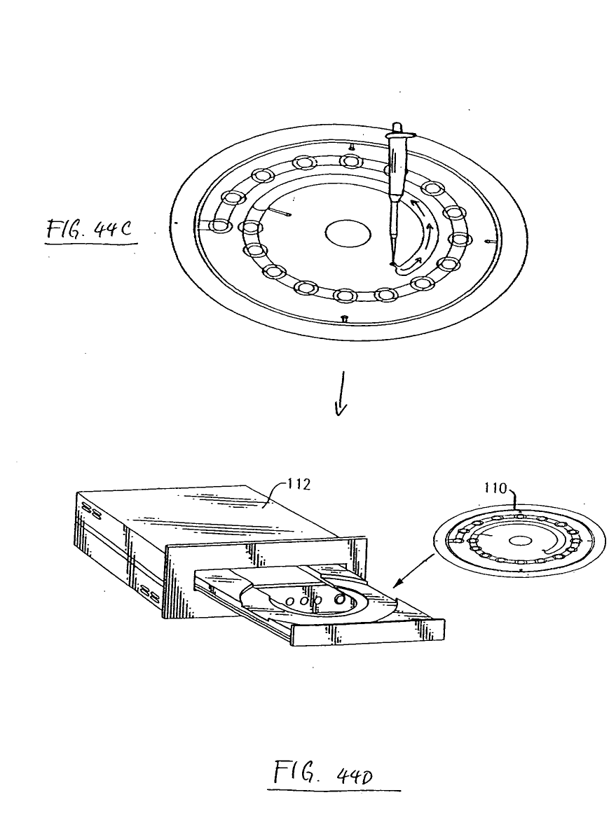

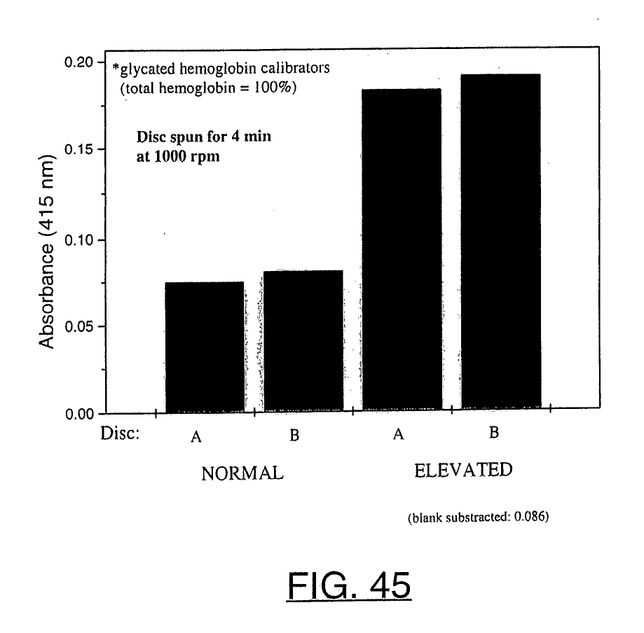

The next set of figures, FIGS. 44A, 44B, 44C, and 44D, shows steps for a method of using the optical bio-disc made as described in conjunction with FIGS. 43A and 43B. FIG. 44A illustrates the loading of a sample into the mixing chamber 134 using a pipette 366. Once the sample is loaded, the disc 110 is loaded into the disc drive 112 and rotated at a pre-determined speed and duration to allow the sample to move through the fluidic circuit at a rate that allows ample time for the analytes present in the sample to bind with their respective capture agents in the bio-matrix pads 654. Each of the bio-matrix pads 654 may contain different types of capture agents to capture different analytes in the sample. The disc 110 is then removed from the drive 112 and signal agents with or without reporters attached thereto are loaded into the mixing chamber 134 (FIG. 44B). These signal agents bind to the captured analytes, if present, in the bio-matrix pads 654. The signal agents may include antigens, antibodies, ligands, receptors, binding agents, DNA, RNA, any molecule that can bind to the target or analyte, or any molecule in which the analyte specifically binds to. While the reporters may include any molecule or material detectable by the optical disc drive 112 or molecules that produce a detectable signal in the presence of the analyte or a substrate. The reporters may be for example, nanopheres, microspheres, fluorescent particles, chemiluminscent particles, phosphorescent particles, enzymes, and enzyme substrates. The next step in the assay is the washing of the analysis zones located within the bio-matrix pads 654 in the inlet 626 and outlet 628 passages. Washing is performed by flushing the bio-matrices with wash buffer by loading the mixing chamber with wash buffer and spinning the disc 110 (FIG. 44C). This washing step may be repeated several times depending on the assay. Washing steps may also be added to any of the intermediate steps described above. The disc is then loaded into the optical disc drive 112 as shown in FIG. 44D. The final step in the assay is the analysis of the bio-matrix pads by directing a beam of electromagnetic radiation from the optical disc drive 112 through the inlet and outlet passages containing the bio-matrix pads to determine whether any reporters are present therein and determine the amount of reporters that are present in each of the bio-matrix pads 654 allowing for the quantitation of the analytes of interest. The spiral fluidic circuit configuration allows for detection and quantitation of multiple analytes within a sample simultaneously since different capture agents and signal agents may be used or placed in the bio-matrix pads 654.

More particular discussion of membranes as implemented on optical bio-discs are provided in the following

### EXAMPLES

### Example 1

### In-Disc Hemoglobin Separation

Weak cation exchange membranes (Vivapure from Vivascience, Hannover, Germany) were embedded in the optical bio-disc as described above in conjunction with FIGS. 41A-41C. Hemoglobin standard solutions (Eagle Diagnostics, De Soto, Tex.) were prepared by reconstituting lyophilized hemoglobin standards containing normal and elevated glycohemoglobin with 1 ml deionized water. The normal standard contained 7% glycohemoglobin while the elevated standard contained 14% glycohemoglobin. Eight and a half microliter aliquots of each of the standard solutions were mixed with 1 ml of 50 mM MOPS [3-(N-morpholino) propane sulfonic acid] buffer, pH 6.9. Two-hundred-fifty microliters of each of the MOPS/glycohemoglobin standard mixtures were loaded into different bio-discs without pre-equilibration (2 discs for the normal standard and 2 discs for the elevated standard). The discs were then rotated at 1000 rpm for 4 minutes. Fifty microliters of each of the filtrate in the circumferential analysis chambers from each of the bio-discs used in this experiment was collected from the vent port. The aliquots of filtrate were then placed in a microtiter plate and analyzed spectrophotometrically at 415 nm twice. The amount of total hemoglobin was also analyzed by taking 50 ul aliquots of the unfiltered MOPS/glycohemoglobin standard mixtures and spectrophotometrically analyzing each mixture at 415 nm. Results from this experiment are shown in FIG. 45 which indicates the successful filtering out of the non-glycated hemoglobin in the sample leaving only the glycated hemoglobin or glycohemoglobin for analysis. FIG. 45 further demonstrates the linearity of the assay as indicated by the ratio of the data collected from the normal (7%) and elevated (14%) standards.

Concluding Summary

All patents, patent applications, technical specifications, and other publications mentioned in this specification are incorporated herein in their entireties by reference.

While this invention has been described in detail with reference to certain preferred embodiments, it should be appreciated that the present invention is not limited to those precise embodiments. Rather, in view of the present optical bio-system disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

1. An optical bio-disc, comprising:
a cap portion having inlet and vent ports formed therein;
a first channel layer having cut-out portions;
a second channel layer having cut-out portions;
a third channel layer having cut-out portions;
a fourth channel layer having cut-out portions; and
a substantially circular substrate having a center and an outer edge, wherein the substrate is configured to support the cap portion, the first channel layer, the second channel layer, the third channel layer, and the fourth channel layer.

2. The optical bio-disc according to claim 1 wherein said cut-out portions in said first channel layer include at least one of an extended arcuate cut-out, short arcuate cut-outs, an inlet channel cut-out, a radially directed cut-out, and a circumferential cut-out.

3. The optical bio-disc according to claim 1 wherein said cut-out portions in said second channel layer include at least one of an extended arcuate cut-out, dumbell shaped cut-outs, an inlet channel cut-out, a radially directed cut-out with a circular cut-out, and a circumferential cut-out.

4. The optical bio-disc according to claim 1 wherein said cut-out portions in said third channel layer include at least one of an extended arcuate cut-out, dumbell shaped cut-outs, a radially directed cut-out with a circular cut-out, and a circumferential cut-out.

5. The optical bio-disc according to claim 1 wherein said cut-out portions in said fourth channel layer include at least one of an extended arcuate cut-out, short arcuate cut-outs, an inlet channel cut-out, and a circumferential cut-out.

6. The optical bio-disc according to any of claims 1, wherein said cut-out portions are in register with each other such that when the bio-disc is assembled a spiral fluidic circuit is formed having an inlet port, a mixing chamber, upper flow chambers, lower pass through chambers, inlet passages, outlet passages, a circumferential analysis chamber, and vent ports in fluid communication.

7. The optical bio-disc according to claim 1 further comprising a chemically modified membrane placed in one or more of the inlet and outlet passages.

8. The optical bio-disc according to claim 1 further comprising biological matrix placed in one or more of the inlet and outlet passages.

9. A method of making a chromatographic optical bio-disc, said method comprising the steps of:
providing a substrate having a center and an outer edge;
providing a cap portion having an inlet port and a vent port formed therein;
providing a first channel layer having cut-out portions;
providing a second channel layer having cut-out portions;
providing a third channel layer having cut-out portions;
providing a fourth channel layer having cut-out portions; and
assembling the optical bio-disc such that said cap portion and said channel layers are supported by the substrate and said cut-out portions form a spiral fluidic circuit.

10. The method according to claim 9 wherein said cut-out portions in said first channel layer include at least one of an extended arcuate cut-out, short arcuate cut-outs, an inlet channel cut-out, a radially directed cut-out, and a circumferential cut-out.

11. The method according to claim 9 wherein said cut-out portions in said second channel layer include at least one of an extended arcuate cut-out, dumbell shaped cut-outs, an inlet channel cut-out, a radially directed cut-out with a circular cut-out, and a circumferential cut-out.

12. The method according to claim 9 wherein said cut-out portions in said third channel layer include at least one of an extended arcuate cut-out, dumbell shaped cut-outs, a radially directed cut-out with a circular cut-out, and a circumferential cut-out.

13. The method according to claim 9 wherein said cut-out portions in said fourth channel layer include at least one of an extended arcuate cut-out, short arcuate cut-outs, an inlet channel cut-out, and a circumferential cut-out.

14. The method according to any of claims 9, wherein said cut-out portions are in register with each other such that when the bio-disc is assembled a spiral fluidic circuit is formed having an inlet port, a mixing chamber, upper flow chambers, lower pass through chambers, inlet passages, outlet passages, a circumferential analysis chamber, and vent ports in fluid communication.

15. The method according to claim 14 further comprising the step of placing a bio-matrix pad over said lower pass through chambers.

16. The method according to claim 14 further comprising the step of placing a chemically modified membrane over said lower pass through chambers.

17. The method according to claim 9 further comprising the step of encoding information on an information layer associated with the substrate, the encoded information being readable by a disc drive assembly to control rotation of the disc.

18. The method according to claim 9 further comprising the step of attaching one or more capture agents onto the optical bio-disc.

19. The method of claim 18 wherein said one or more capture agents is selected from the group comprising antigen, antibody, ligand, receptor, binding agents, DNA, RNA, any molecule that can bind to the target or analyte, and any molecule in which the analyte specifically binds to.

20. A method of using an optical bio-disc, the method comprising:
depositing a test sample into the bio-disc through an inlet port;
rotating said bio-disc at a predetermined speed and for a predetermined period of time to allow said test sample to move through a bio-matrix pad so that analytes present in the sample bind to capture agents in the bio-matrix pad;
continuing said rotating step to thereby move said test sample through a spiral fluidic circuit of the optical bio-disc and into an analysis chamber;
depositing signal agents having one or more reporters attached thereto into the bio-disc through said inlet port;
rotating said disc to cause said signal agents to move through said bio-matrix pad so that said signal agents bind to any analyte that is bound to the capture agents in the bio-matrix pad; and
scanning the bio-matrix pads located in the inlet and outlet passages with a beam of electromagnetic radiation to determine the presence and amount of signal agents bound to the analytes within the bio-matrix pads.

21. The method according to claim 20 further comprising the step of calculating the amount of analyte present in the sample based on the amount of bound signal agents.

22. The method of claim 20 wherein said signal agents are selected from the group comprising antigens, antibodies, ligands, receptors, binding agents, DNA, RNA, any molecule that can bind to the target or analyte, and any molecule in which the analyte specifically binds to.

23. The method of claim 20 wherein said one or more reporters is selected from the group comprising any molecule or material detectable by an optical disc drive, and any molecule that produces a detectable signal in the presence of the analyte or a substrate.

24. The method of claim 20 wherein said one or more reporters is selected from the group comprising nanopheres, microspheres, fluorescent particles, chemiluminscent particles, phosphorescent particles, enzymes, and enzyme substrates.
